SEQUENCE LISTING (144 sequences)

SEQ ID NO: 1 - DNA, 231 bp, Homo sapiens
gcctcgggcaaactctttggacagacagaccttccaagagggccctcaggctccgcacgtggtctgggcgggaagtgcgggcaagagactaggaagccttcccagcagcgccggcggaccggggcaaggaaagggtcgaatagttaccatgccattctggggccgtccctctgattggcccgagctccctcagcccgagtcgcggcgctccaaggaagggggcggagtctc

SEQ ID NO: 2 - DNA, 20 bp, Artificial sequence
  Primer
gcctcgggcaaactctttgg

SEQ ID NO: 3 - DNA, 20 bp, Artificial sequence
  Primer
gagactccgcccccttcctt

SEQ ID NO: 4 - DNA, 246 bp, Homo sapiens
tgcaaaggcggtttcacctgggcgcggaaaggctgcatgacctccggcagctccgggcttagtaccagggactggactggggggacacgaccctacggattcgcgccgtcgcccacctggccaggccccatgtgcgcccctagccaccagggcgcgaggggcggcaggggcggggccttggggaagcggcctaggggtggtccctgggagtttccatgggcggggcctgctggggagatgcgattg

SEQ ID NO: 5 - DNA, 20 bp, Artificial sequence
  Primer
tgcaaaggcggtttcacctg

SEQ ID NO: 6 - DNA, 20 bp, Artificial sequence
  Primer
caatcgcatctccccagcag

SEQ ID NO: 7 - DNA, 203 bp, Homo sapiens
tctggttctcgctggggttgtcgcggggcctgtgacaccagatcgttttctgcccacagctgatgaagcgagtgtataagggcattcccatgaacgtccgggccgaggtgtggtcagtcctcctgaacattcaggaaatcaaggcgaaaaaccccagacaataccgggtatgctcagccacagcacaacaaacaggccaggcc

SEQ ID NO: 8 - DNA, 20 bp, Artificial sequence
  Primer
tctggttctcgctggggttg

SEQ ID NO: 9 - DNA, 20 bp, Artificial sequence
  Primer
ggcctggcctgtttgttgtg

SEQ ID NO: 10 - DNA, 231 bp, Homo sapiens
cctccgtgctcactgctggtgttgcctcttggaacctcggcagtttctgctttcgcacctgcagggtttagtctccgggactgcttgggaggacacctggaatgccgaaggcaggactacacaccgggcgaatccaccagcgccaccaagcccttgaaaacccgaggccgccgcgcgactccatttcccagcgccccgcgcggcaggggcttggacgttgccaaggagacc

SEQ ID NO: 11 - DNA, 20 bp, Artificial sequence
  Primer
cctccgtgctcactgctggt

SEQ ID NO: 12 - DNA, 20 bp, Artificial sequence
  Primer
ggtctccttggcaacgtcca

SEQ ID NO: 13 - DNA, 336 bp, Homo sapiens
cgcctttcccacacactgcttcttctgtttttacctcgacttccttcctattggttcccttcgtcctccggagttggcacctacggctgttgattggcttttctggacgcccattgtgcccaccctcataccacgtggcattcctagcgctaattgggcaattccactacctcgtttatcaggagttgacggttgactggctactctgagggagggcttgagggaggggcgtggccagccgagaccccgcccccaacccccctgctgggccgggtaggcgtttcagtctttcgcggcccgggagctcagcagagctaccagctgccctgttggctt

SEQ ID NO: 14 - DNA, 20 bp, Artificial sequence
  Primer
cgcctttcccacacactgct

SEQ ID NO: 15 - DNA, 20 bp, Artificial sequence
  Primer
aagccaacagggcagctggt

SEQ ID NO: 16 - DNA, 223 bp, Homo sapiens
cgggagtgaagggagggtgtggcccgcgggtgggatctgtagagcagacaaaatatggggcccctggcgcttaaagttcagtttgtctctcttgagcttggagaaaatcatccgtagtgcctccccgggggacacgtagaggagagaaaagcgaccaagataaaagtggacagaagaataagcgagactttttatccatgaaacagtctcctgccctcgctcc

SEQ ID NO: 17 - DNA, 20 bp, Artificial sequence
  Primer
cgggagtgaagggagggtgt

SEQ ID NO: 18 - DNA, 20 bp, Artificial sequence
  Primer
ggagcgagggcaggagactg

SEQ ID NO: 19 - DNA, 204 bp, Homo sapiens
caggggaaggcccagaaatgctcctgaagcatcttgctgtcaccagtgcccctgctgaggccccagagggcaggtgacctgggtgaacctcctaacggacagggggcttctccgggccctgggtcccgggtgcccgctcccacccccttagcagaattttggagagtgtggctgtgtttccttcgtggttgtttggacagcggc

SEQ ID NO: 20 - DNA, 20 bp, Artificial sequence
  Primer
caggggaaggcccagaaatg

SEQ ID NO: 21 - DNA, 20 bp, Artificial sequence
  Primer
gccgctgtccaaacaaccac

SEQ ID NO: 22 - DNA, 236 bp, Homo sapiens
tcccactggccaaggagaaaagagcaaggtcacttgtcggggggctgcagagggaattaccttctttcatttgcaaatgttactgggggacacaccggctcccagtagggtttccgccaaggctccgcgaaacgccactagagggcgccgctagcgaatcccacagcgcgccccgctgcccccacttttgtcctccgggttcacacgggcgcccggaagagagggtggtgcctggg

SEQ ID NO: 23 - DNA, 20 bp, Artificial sequence
  Primer
tcccactggccaaggagaaa

SEQ ID NO: 24 - DNA, 20 bp, Artificial sequence
  Primer
cccaggcaccaccctctctt

SEQ ID NO: 25 - DNA, 196 bp, Homo sapiens
agccctggggcaagaacaagcaccgccccctttcctgtaaatgctctggagtccgccagacacccattctccggaggaggaaactgaggcacagagagaaaggcacctgcccaaggtcacagagccaaggctcgatcgggagccccaggaggggtcccggggccaccctggtagcggagaagaccgcacgctgagc

SEQ ID NO: 26 - DNA, 20 bp, Artificial sequence
  Primer
agccctggggcaagaacaag

SEQ ID NO: 27 - DNA, 20 bp, Artificial sequence
  Primer
gctcagcgtgcggtcttctc

SEQ ID NO: 28 - DNA, 350 bp, Homo sapiens
gaggcaggtccagattcccttcggaggctctaattggccttcttgacagtcactcgcctgtatctggcttacgattggtctttgggggacgaggactaggcaccccctgcttgcggtccctcggcccgttttcctgctagtggagtgccggtgtcccctcctcttggttggacctgattggctccacctcccgatctgagcattgtgattgggtgcagctgcgctgggcgggacggcctggagcgtcggccgttggcgagcgctctatccttgttcccctcccttctctcgtcagactccggcgccggagctccacccccactgacgggttctgattggccgcttctgac

SEQ ID NO: 29 - DNA, 22 bp, Artificial sequence
  Primer
gaggcaggtccagattcccttc

SEQ ID NO: 30 - DNA, 22 bp, Artificial sequence
  Primer
gtcagaagcggccaatcagaac

SEQ ID NO: 31 - DNA, 208 bp, Homo sapiens
ccatgtttcccccagcttcccgttcccagggccctgggtggggcgcgccccatatcccccacccactttcctccttctctccccctccacgccgccgcgcacattccaaccccaggctcctgcgatcccggcaggccaaaaagtctggagcggataaatagccacaagatccggagtcgctcgccgtagctctggtccaccacccaga

SEQ ID NO: 32 - DNA, 20 bp, Artificial sequence
  Primer
ccatgtttcccccagcttcc

SEQ ID NO: 33 - DNA, 20 bp, Artificial sequence
  Primer
tctgggtggtggaccagagc

SEQ ID NO: 34 - DNA, 223 bp, Homo sapiens
accgcgcctggcctaattttgcatttttaagtagagacggggtttcaccatgtgggcaaggctagtctcgaactcctgacttcgtgatctgcccgcctcggcctcccaaaatgctgggattacaggcataagccaccgtgcccggcttgagataatgattcttaacatgtggtatgtagagcaagtgtactcccgccctagactgtgagccccgtgagagatg

SEQ ID NO: 35 - DNA, 19 bp, Artificial sequence
  Primer
accgcgcctggcctaattt

SEQ ID NO: 36 - DNA, 20 bp, Artificial sequence
  Primer
catctctcacggggctcaca

SEQ ID NO: 37 - DNA, 253 bp, Homo sapiens
tcacgatggccagctccttccactgccgcccaggtgccctcccaggctcctgctgcccttcgcccagtccctccggtggtcagtgccagcatgtagatcggtggcttaaacctgcactgttgcaggagtgcctgggatctgtgccaagagagcttgatccctggaggcagccacagagtgggtccccctgtccttggcctgccacgacccatcaatcatcccggatgcaatggcattcagccctgcctggggt

SEQ ID NO: 38 - DNA, 20 bp, Artificial sequence
  Primer
tcacgatggccagctccttc

SEQ ID NO: 39 - DNA, 19 bp, Artificial sequence
  Primer
accccaggcagggctgaat

SEQ ID NO: 40 - DNA, 183 bp, Homo sapiens
atcgagcaagctcggggaacgtgaccgggggctgcatgcgtcagctaacagaacagaaagttttgcagtgctttctcatacaatgtctggaatttacagataacacaagtagtttaggtcaggggttgatattattatcacttttttttaactaccagggccaggtggtggcgccaaggtctt

SEQ ID NO: 41 - DNA, 20 bp, Artificial sequence
  Primer
atcgagcaagctcggggaac

SEQ ID NO: 42 - DNA, 19 bp, Artificial sequence
  Primer
aagaccttggcgccaccac

SEQ ID NO: 43 - DNA, 239 bp, Homo sapiens
gccgtggcaaaatgagctgtcaactttaggttgacaggggtgtggccgcgaccgcaagggcttttgttgccgggtggacccaacagggatgggctgctggggacagctgctggtgtggttcggagccgcgggtaagtgctatctggcggtcaggggaccgtccagcctgaggcactttcctaggcttgggggtggccggtcaacaggccaggtgtcagtggttcctccagaggcctcga

SEQ ID NO: 44 - DNA, 20 bp, Artificial sequence
  Primer
gccgtggcaaaatgagctgt

SEQ ID NO: 45 - DNA, 20 bp, Artificial sequence
  Primer
tcgaggcctctggaggaacc

SEQ ID NO: 46 - DNA, 208 bp, Homo sapiens
tggggttactgggacggtgagtaactgccggcccccgtccgcaatcgggctccctccgtcgggcgcgagggggcaccccagggctggggggactaggttcctccaaccccggggggcccctacacccagccctgggctcccgattgcggtcccatcaccccctcctggcggcagaagtctcccttgcaatgtttcaggcggggacctc

SEQ ID NO: 47 - DNA, 20 bp, Artificial sequence
  Primer
tggggttactgggacggtga

SEQ ID NO: 48 - DNA, 20 bp, Artificial sequence
  Primer
gaggtccccgcctgaaacat

SEQ ID NO: 49 - DNA, 308 bp, Homo sapiens
gcgagggctcgttctgggtagaggcccaaatacatttaacctgtgtagccagaaggtagagtaggaccagtgagggctagtgagggaggtagacttgcacgtcatgaaatgaaaaactttctaaaagccggcgtttccaaagccccaacaaccgctcttggccaggtgatgagcgccctgttcctggaggtatgtagaacacgcatttgaaggcgccaaggtacaagggattcaggcatcggatgggacagaggccgggtgtccctgggtcaccgtccctgagagcgctgtacgggagctaggcgtgg

SEQ ID NO: 50 - DNA, 20 bp, Artificial sequence
  Primer
gcgagggctcgttctgggta

SEQ ID NO: 51 - DNA, 20 bp, Artificial sequence
  Primer
ccacgcctagctcccgtaca

SEQ ID NO: 52 - DNA, 318 bp, Homo sapiens
cgtcgaagccaggtcttgagcgtcagacagaatgaggtgtcccagagcggcggaggagagccagacgcacgctggttccggtctggacggaatcgccgcggagcacggcagaggctagggcggaatggctacggcagcgcagttcgccaaggctcggtctccgccctgcagcctatttcctctaacccgcagatccactatgggaggaggcggggagcgggctggagagcactaacagagacaggcggggcgagtcgtgggcgcgtgacgtcaccccggggtgtgcgcggcgcgagcggaagcggaagcggctctgtt

SEQ ID NO: 53 - DNA, 21 bp, Artificial sequence
  Primer
cgtcgaagccaggtcttgagc

SEQ ID NO: 54 - DNA, 20 bp, Artificial sequence
  Primer
aacagagccgcttccgcttc

SEQ ID NO: 55 - DNA, 180 bp, Homo sapiens
ctccaaagcccacgctaccaggtacaacctcaaggctgcggcgtctcttcacctgccccctagcccccaaaccgctgctatgtctagggcctgacattccggcgccctctgggacgtgctcagatgcaggggcgcaaacgccaaaggagaccaggctgtaggaagagaagggcagagcgc

SEQ ID NO: 56 - DNA, 20 bp, Artificial sequence
  Primer
ctccaaagcccacgctacca

SEQ ID NO: 57 - DNA, 20 bp, Artificial sequence
  Primer
gcgctctgcccttctcttcc

SEQ ID NO: 58 - DNA, 155 bp, Homo sapiens
acgtcgaccccctagcgagagggagcgacgggggcggtgccgcggggctcctgagtggcggatgcgagggacggggcggggccaatgccggcgtgccactttctgattggtaggttttggggtcccgcccctgagaggagggcaaggccatggta

SEQ ID NO: 59 - DNA, 20 bp, Artificial sequence
  Primer
acgtcgaccccctagcgaga

SEQ ID NO: 60 - DNA, 20 bp, Artificial sequence
  Primer
taccatggccttgccctcct

SEQ ID NO: 61 - DNA, 226 bp, Homo sapiens
caggcaggcagatgttgacaaagagggctctccaaaaaccatgttcggatagatttttgcgaactgcacagataaataggagcagaaggccggtcacctctgtaaccagcggtagcagcagcagaagccgcagcttcagaggcagccggagagacctcggagcagagaaggcgccgccgaccctcgcggctgcctggcccgcggctcctacaaaggcgggctagcc

SEQ ID NO: 62 - DNA, 22 bp, Artificial sequence
  Primer
caggcaggcagatgttgacaaa

SEQ ID NO: 63 - DNA, 22 bp, Artificial sequence
  Primer
ggctagcccgcctttgtaggag

SEQ ID NO: 64 - DNA, 309 bp, Homo sapiens
ttctcctgcctccagctcctctctggacccctgtcctggcacctcttcggtccctggttcggtctgcccctttcccaccgcggcccgtcttaggccaggatgtgctccctgccctgcggactctggagcagggcccggccactccccggagcctgtatgacgggaaccgccccgcgccctctcccctacgcggggcaggccagccctggggcgccttaaaaaccggagctggcgcttggcatcgccactctgggcaggatccaacgtcgctccagctgctcttgacgactccacagataccccgaagcc

SEQ ID NO: 65 - DNA, 22 bp, Artificial sequence
  Primer
ttctcctgcctccagctcctct

SEQ ID NO: 66 - DNA, 22 bp, Artificial sequence
  Primer
ggcttcggggtatctgtggagt

SEQ ID NO: 67 - DNA, 250 bp, Homo sapiens
ctttggggtccaggcaggttttggggcctcctgtctggtgggaggaggccgcagcgcagcaccctgctcgtcacttgggatggagaccggctttcccgcaatcatgtaccctggatcttttattgggggctggggagaagagtatctcagctgggaaggaccggggctcccagatttcgtcttccaggtaacgtgggtttagtatcccgacttggaggcttgtcagaatgtttctctccttccagcccaa

SEQ ID NO: 68 - DNA, 22 bp, Artificial sequence
  Primer
ctttggggtccaggcaggtttt

SEQ ID NO: 69 - DNA, 22 bp, Artificial sequence
  Primer
ttgggctggaaggagagaaaca

SEQ ID NO: 70 - DNA, 200 bp, Homo sapiens
gggccagctgctgttctccttaataacgagaggggaaaaggagggagggagggagagattgaaaggaggaggggaggaccgggaggggaggaaaggggaggaggaaccagagcggggagcgcggggagagggaggagagctaactgcccagccagcttgcgtcaccgcttcagagcggagaagagcgagcaggggagagc

SEQ ID NO: 71 - DNA, 22 bp, Artificial sequence
  Primer
gggccagctgctgttctcctta

SEQ ID NO: 72 - DNA, 22 bp, Artificial sequence
  Primer
gctctcccctgctcgctcttct

SEQ ID NO: 73 - DNA, 203 bp, Homo sapiens
accctctcgacacccttgatccgagtccagatctgcactagcaaccagaactaatatttcatttaaccccaccaaagggggaggcgagaggagccagaagcaaacttcagctgtctcagccggatccgtggttcctacatttggaggagccgcgtgccagaaggcgtaggaccccaaggggggacaaggaggactcccgagtc

SEQ ID NO: 74 - DNA, 22 bp, Artificial sequence
  Primer
accctctcgacacccttgatcc

SEQ ID NO: 75 - DNA, 22 bp, Artificial sequence
  Primer
gactcgggagtcctccttgtcc

SEQ ID NO: 76 - DNA, 201 bp, Homo sapiens
gaggtggggacgagagttgagctctcaccgccctctgcacactcgagaacgaggaccctgcaattgagcacaagcatgctgcatgggggcgcaccccagcctctccgcgcgcgccgggaggccccccagccaacatgagttacaccggcgattacgtgctttcggtgagaacaccgagtgacgatctgttgcttcccctga

SEQ ID NO: 77 - DNA, 22 bp, Artificial sequence
  Primer
gaggtggggacgagagttgagc

SEQ ID NO: 78 - DNA, 22 bp, Artificial sequence
  Primer
tcaggggaagcaacagatcgtc

SEQ ID NO: 79 - DNA, 213 bp, Homo sapiens
aagtccctttctcggtgggagactgaggccgccttggcggggcgggacgagactcctccgaggtcgggaaagggggccccgcagcagccccttggcttcccttctcccttgcctcccctccggggctccggttcagaggcactctgggcgcctgctacagcttccaaactgcgccgcttccttcttcggcagaaaaggactttcagatgcggc

SEQ ID NO: 80 - DNA, 22 bp, Artificial sequence
  Primer
aagtccctttctcggtgggaga

SEQ ID NO: 81 - DNA, 23 bp, Artificial sequence
  Primer
gccgcatctgaaagtccttttct

SEQ ID NO: 82 - DNA, 245 bp, Homo sapiens
ccaaggccacgtctctacgcttccgaacagcgagttcttgatcatgcccaacatgttgtagccgagacgctcccgacgcacgggaggacgtgaggtggcgggggcgacggagcaccacgggcagcgaccaccggcggcagggcggcagggcggcagggcggcagggcggcagggtggcagggcggcaaggcggcgggacggcgaggcggcgaggcgagaggcggggctagaggcaggggccagag

SEQ ID NO: 83 - DNA, 20 bp, Artificial sequence
  Primer
ccaaggccacgtctctacgc

SEQ ID NO: 84 - DNA, 20 bp, Artificial sequence
  Primer
ctctggcccctgcctctagc

SEQ ID NO: 85 - DNA, 219 bp, Homo sapiens
gtgctttttcctccccttgagcgcctctcttttctctttttggtcccgtttcgccccgatctcgctctctttttgctccgggtttccctccgactggccctcgaaaggcgcctgaatccgtgtcaatatagctgcttcaatttcgccgcgcgtgtcaggcgggcgggcgggcgggtgctcaccgcgctcggggttttcttttcttcaaccaccctccgc

SEQ ID NO: 86 - DNA, 22 bp, Artificial sequence
  Primer
gtgctttttcctccccttgagc

SEQ ID NO: 87 - DNA, 22 bp, Artificial sequence
  Primer
gcggagggtggttgaagaaaag

SEQ ID NO: 88 - DNA, 204 bp, Homo sapiens
ctgtggcggtggtccagttccgccaggaaaccgccgcctggagctgtgggtcgcgcacattaacgcatccagcggaaaaatgaaggagacccaaattcaaagttaaagtaatggtgacccgagaggtgccttgatgagaaggtttggggtcccggttactgatggttatcattcttacgagatgctggtcacctacgaagggag

SEQ ID NO: 89 - DNA, 20 bp, Artificial sequence
  Primer
ctgtggcggtggtccagttc

SEQ ID NO: 90 - DNA, 22 bp, Artificial sequence
  Primer
ctcccttcgtaggtgaccagca

SEQ ID NO: 91 - DNA, 200 bp, Homo sapiens
cactgacacaggggtcacgaaaatgctaaaacaagacgtgcaagaattgttgcgctccgcacacgtaacaagggctctgcctccccctcccccaatcttcgccctggtggccaggccctgggctccgccccttccccaccagaccgggggaggggtcctcctcggcatggaggtaggggatgctaggctgctggagacgc

SEQ ID NO: 92 - DNA, 22 bp, Artificial sequence
  Primer
cactgacacaggggtcacgaaa

SEQ ID NO: 93 - DNA, 21 bp, Artificial sequence
  Primer
gcgtctccagcagcctagcat

SEQ ID NO: 94 - DNA, 234 bp, Homo sapiens
aaagggcaaggcttggaattgaacccaggaagcttcctggaggaggaggcggggctggcttggtagaacaaagggaggggtggattttcttgctggtgaaaagagattgagtgggaggttcagggaagagtgcaaacatccttcccctctctccccctagccctggggctccttctctgctccttcccggccgggtttgggggcgctcgggagggtgacggcagggtcctcgag

SEQ ID NO: 95 - DNA, 22 bp, Artificial sequence
  Primer
aaagggcaaggcttggaattga

SEQ ID NO: 96 - DNA, 20 bp, Artificial sequence
  Primer
ctcgaggaccctgccgtcac

SEQ ID NO: 97 - DNA, 200 bp, Homo sapiens
ctccgagccacatgcaggatataatctcccggtgtgccgttttttaagcccgttggaaaagcgcagtattagggtgggagtgacccgattttccaggtgccgtctgtcgcccctttctttgactcggaaagggaactccctgaccccttgcgcttcccgagtgaggcaatgcctcgccctgcttcagctcgtgcacggtg

SEQ ID NO: 98 - DNA, 20 bp, Artificial sequence
  Primer
ctccgagccacatgcaggat

SEQ ID NO: 99 - DNA, 18 bp, Artificial sequence
  Primer
caccgtgcacgagctgaa

SEQ ID NO: 100 - DNA, 184 bp, Homo sapiens
tgggtccgctcctgtttctgcctccgcgtgagggttgcggccacagggtgacggcgtggtgggcgacacccccgcgcaggcatgcacagagtggtggtcgttgggcgaaggttggtcgtgaggttcttgggcttcaggagcgaggcttggaaccggtgggcgggactgtgtgcagaggtggggc

SEQ ID NO: 101 - DNA, 20 bp, Artificial sequence
  Primer
tgggtccgctcctgtttctg

SEQ ID NO: 102 - DNA, 20 bp, Artificial sequence
  Primer
gccccacctctgcacacagt

SEQ ID NO: 103 - DNA, 175 bp, Homo sapiens
cagtgtgttggcgcgtgttcgagtacagatacaccgggggtgtttgggtacccgcacatggctgcgggtggggcgcagtggagaggaagcacacatgcgtgtgctgagatatggccgcatccttgtgctcccccagcccagacgcaggggagaccagcaccgagacacccgagct

SEQ ID NO: 104 - DNA, 20 bp, Artificial sequence
  Primer
cagtgtgttggcgcgtgttc

SEQ ID NO: 105 - DNA, 20 bp, Artificial sequence
  Primer
agctcgggtgtctcggtgct

SEQ ID NO: 106 - DNA, 196 bp, Homo sapiens
ccagcccagcgctgaagtaacgggaccatgcccagtcccaggccccggagcaggaaggctcgagggcgcccccaccccacccgcccaccctccccgcttctcgctaggtccctattggctggcgcgctccgcggctgggatggcagtgggaggggaccctctttcctaacggggttataaaaacagcgccctcggc

SEQ ID NO: 107 - DNA, 20 bp, Artificial sequence
  Primer
ccagcccagcgctgaagtaa

SEQ ID NO: 108 - DNA, 20 bp, Artificial sequence
  Primer
gccgagggcgctgtttttat

SEQ ID NO: 109 - DNA, 215 bp, Homo sapiens
cctgcctcccgacactcttggcgaggtttttgtacagtttgctccgggagctgtttcttcgcttccacctttttctcccccacacttcgcggcttcttcatgctttttcttctcaccatttctggccaaaactacaaacaagacttcgcaggtaggttttttttcctccccttttctctctttttatccctttttggtgtgctcgtcctccatcc

SEQ ID NO: 110 - DNA, 20 bp, Artificial sequence
  Primer
cctgcctcccgacactcttg

SEQ ID NO: 111 - DNA, 20 bp, Artificial sequence
  Primer
ggatggaggacgagcacacc

SEQ ID NO: 112 - DNA, 249 bp, Homo sapiens
caccctctgtggccctgtgtcctgtaggtattgggagatccacagccaagatgccgggaccccttagaagcctagaaatggtgagagtgcccattggacatcctgagagaggggagggactggttgatgggaagtggctgtggagggactcaggcctccccgtagtcagctccacaatctgcgtccggacttctccttacccagctcggcctcagtccccttcagccataagatggtggctgcgctgac

SEQ ID NO: 113 - DNA, 20 bp, Artificial sequence
  Primer
caccctctgtggccctgtgt

SEQ ID NO: 114 - DNA, 20 bp, Artificial sequence
  Primer
gtcagcgcagccaccatctt

SEQ ID NO: 115 - DNA, 245 bp, Homo sapiens
tgagtttgctgcgtgagtaccgcccgcgcgccgcggccgcttggcttcgccgcggggagggtggaggctttctgggaggctgaacagcagagcagagtctcacggagggaagggacccctgcccaacccacgcactgccgcccacagctgcttccccccggggccagcgcctcacctgggagctgacgggggtgggaggggaagggaaggccatcacccccgcgagtgtgcgttagccgaggtgt

SEQ ID NO: 116 - DNA, 22 bp, Artificial sequence
  Primer
tgagtttgctgcgtgagtaccg

SEQ ID NO: 117 - DNA, 22 bp, Artificial sequence
  Primer
acacctcggctaacgcacactc

SEQ ID NO: 118 - DNA, 282 bp, Homo sapiens
acaaaggtcaggctgggaggagggcacagccaggctcgggctcagcttggggtgggggctccgtggggctggcgcctctcccgggtcaggtgctgagccgatgctggccggcgtaggcctctttcccagtttctctggggggcaggagcaccttgccaacctctgatggctccagcgcctgcactcgcctcacaccacatggtgacttcaacggggaccagggaagccctcgcccctcccacccctcagagccctccccgacctcgcccacttctcagagct

SEQ ID NO: 119 - DNA, 22 bp, Artificial sequence
  Primer
acaaaggtcaggctgggaggag

SEQ ID NO: 120 - DNA, 22 bp, Artificial sequence
  Primer
agctctgagaagtgggcgaggt

SEQ ID NO: 121 - DNA, 161 bp, Homo sapiens
gtgagaaacagcaggcgctagggtttagaggaggcctggggctgaggtttcagggacctgggctcagggcttagatcaccggttcgagtacacccagggggaggactggggtcggggctggggcaggacccctgcgtccactgagtctcgggaaagaatca

SEQ ID NO: 122 - DNA, 22 bp, Artificial sequence
  Primer
gtgagaaacagcaggcgctagg

SEQ ID NO: 123 - DNA, 24 bp, Artificial sequence
  Primer
tgattctttcccgagactcagtgg

SEQ ID NO: 124 - DNA, 231 bp, Homo sapiens
cgccttcttcttcatcggcttttttggaagcatattcttcatcaccgtcatcagcattgataggtacctggccatcgtcctggccgccaactccatgaacaaccggaccgtgcagcatggcgtcaccatcagcctaggcgtctgggcagcagccattttggtggcagcaccccagttcatgttcacaaagcagaaagaaaatgaatgccttggtgactaccccgaggtcct

SEQ ID NO: 125 - DNA, 22 bp, Artificial sequence
  Primer
cgccttcttcttcatcggcttt

SEQ ID NO: 126 - DNA, 22 bp, Artificial sequence
  Primer
aggacctcggggtagtcaccaa

SEQ ID NO: 127 - DNA, 176 bp, Homo sapiens
gccaatcagctgcccaggaagataatgaaaacttgagtaagacaggcagccaaagccacagcggggacgttcccagcctggctcgcttccggcactgacgcctccagccgcgacctctagacttcagcccttccattggtcgtccgtcacagtgccacagtgcgccatgctcacac

SEQ ID NO: 128 - DNA, 20 bp, Artificial sequence
  Primer
gccaatcagctgcccaggaa

SEQ ID NO: 129 - DNA, 20 bp, Artificial sequence
  Primer
gtgtgagcatggcgcactgt

SEQ ID NO: 130 - DNA, 234 bp, Homo sapiens
gctccccaactcagcagagagagcacaccatcagacttctaagacttagtagccaagaagtgttgaattaaactctctgagacctctctttagtctgaccctggcagcctcagtctcccagagcctgtgggaactcggcagccgagaggcagaaggctgggcgacgtccggagaagaagaaacgggggaagaacttttctcttacgatctggctttactctcacgcgcacagcc

SEQ ID NO: 131 - DNA, 22 bp, Artificial sequence
  Primer
gctccccaactcagcagagaga

SEQ ID NO: 132 - DNA, 22 bp, Artificial sequence
  Primer
ggctgtgcgcgtgagagtaaag

SEQ ID NO: 133 - DNA, 265 bp, Homo sapiens
atcgccaagtttggcactgtaatttttttttgagacggagtctcgtactgtcgcccaggctggagtgcagtggtgccatctccactcactgcaagctctgcctcccgggttcacgccattctcctgcctcagcctccagagtagctgggactacaggcgcccgccaccacctctggctaatttttttgtatttttactagagacggggtttcactgtgttagccaggatggtctcgatctcctgacctcgtgatccgccctcctc

SEQ ID NO: 134 - DNA, 20 bp, Artificial sequence
  Primer
atcgccaagtttggcactgt

SEQ ID NO: 135 - DNA, 18 bp, Artificial sequence
  Primer
gaggagggcggatcacga

SEQ ID NO: 136 - DNA, 340 bp, Homo sapiens
tttggagaagacggaggggactagcttacgtcgagacagaggagttcataactattaactatacacaacgacgtgtcacaaatgtttacaaacttccaagagataacgataagaagcggccgggcgcggtggctcacgcctgtaatcccagcactttgggaggccgaggcgggcggatcacgaggtcaggagatcgagaccatcctggctaacacggtgaaaccccgtctctactaaaaatacaaaaaattagccgggcgtggtggcgggcgcctgtagccccagctactcgggaggctgaggcaggagaatggcgtgaacctacctgggaggcggagtt

SEQ ID NO: 137 - DNA, 22 bp, Artificial sequence
  Primer
tttggagaagacggaggggact

SEQ ID NO: 138 - DNA, 22 bp, Artificial sequence
  Primer
aactccgcctcccaggtaggtt

SEQ ID NO: 139 - DNA, 244 bp, Homo sapiens
cgccgatcacacccagtagccctgggctgcagttcggtcctcccagcgcagggggagccatgcgacggcggaagcgggccccggccggcctcctcttcctgcgcccgcgccgccgcgggtggccgcgcgggtgaagccggaagcagcagccaggagggcggggccgcgtagggcccactggccagggagggcgccgcgcggaggcgcggggcgtgtctcctgtcaaaagccatgctcggcaggt

SEQ ID NO: 140 - DNA, 20 bp, Artificial sequence
  Primer
cgccgatcacacccagtagc

SEQ ID NO: 141 - DNA, 20 bp, Artificial sequence
  Primer
acctgccgagcatggctttt

SEQ ID NO: 142 - DNA, 194 bp, Homo sapiens
cccccactccccattcaaagccctcttctctgaagtctccggttcccagagctcttgcaatccaggctttccttggaagtggctgtaacatgtatgaaaagaaagaaaggaggaccaagagatgaaagagggctgcacgcgtgggggcccgagtggtgggcggggacagtcgtcttgttacaggggtgctggcc

SEQ ID NO: 143 - DNA, 20 bp, Artificial sequence
  Primer
cccccactccccattcaaag

SEQ ID NO: 144 - DNA, 20 bp, Artificial sequence
  Primer
ggccagcacccctgtaacaa

(19) United States
(12) Patent Application Publication
(10) Pub. No.: US 2006/0211009 A1
(43) Pub. Date: Sep. 21, 2006

(54) SYSTEM FOR BIOMARKER DISCOVERY

(76) Inventors: Sungwhan An, Daejeon (KR); Chiwang Yoon, Daejeon (KR); Tae Jeong Oh, Daejeon (KR); Myungsoon Kim, Daejeon (KR)

Correspondence Address: JHK LAW, P.O. BOX 1078, LA CANADA, CA, 91012-1078, US

(21) Appl. No.: 11/277,033

(22) Filed: Mar. 20, 2006

Related U.S. Application Data
(60) Provisional application No. 60/594,203, filed on Mar. 18, 2005.

(51) Int. Cl. C12Q 1/68; C07H 21/04

(52) U.S. Cl. 435/6; 536/25.3

(57) ABSTRACT

The present application discloses a method for discovering a methylation marker gene for the conversion of a cell comprising: (i) comparing converted and unconverted cell gene expression content to identify a gene that is present in greater abundance in the unconverted cell; (ii) treating a converted cell with a demethylating agent and comparing its gene expression content with gene expression content of an untreated converted cell to identify a gene that is present in greater abundance in the cell treated with the demethylating agent; and (iii) identifying a gene that is common to the identified genes in steps (i) and (ii), wherein the common identified gene is the methylation marker gene.

### BACKGROUND OF THE INVENTION

1. Field of the Invention

The invention relates to a systematic approach to discovering biomarkers in cell conversion. The invention relates to discovering cancer biomarkers including cervical cancer and its stages. The invention further relates to diagnosis and prognosis of cancer using the biomarkers.

2. General Background and State of the Art

Despite the current developed state of medical science, five-year survival rate of human cancers, particularly solid cancers (cancers other than blood cancer) that account for a large majority of human cancers, are less than 50%. About two-thirds of all cancer patients are detected at a progressed stage, and most of them die within two years after the diagnosis of cancer. Such poor results in cancer diagnosis and therapy are due not only to the problem of therapeutic methods, but also to the fact that it is not easy to diagnose cancer at an early stage or to accurately diagnose progressed cancer or observe it following therapeutic invention.

In current clinical practice, the diagnosis of cancer typically is confirmed by performing tissue biopsy after history taking, physical examination and clinical assessment, followed by radiographic testing and endoscopy if cancer is suspected. However, the diagnosis of cancer by the existing clinical practices is possible only when the number of cancer cells is more than a billion, and the diameter of cancer is more than 1 cm. In this case, the cancer cells already have metastatic ability, and at least half thereof have already metastasized. Meanwhile, tumor markers for monitoring substances that are directly or indirectly produced from cancers, are used in cancer screening, but they cause confusion due to limitations in accuracy, since up to about half thereof appear normal even in the presence of cancer, and they often appear positive even in the absence of cancer. Furthermore, the anticancer agents that are mainly used in cancer therapy have the problem that they show an effect only when the volume of cancer is small.

The reason why the diagnosis and treatment of cancer are difficult is that cancer cells are highly complex and variable. Cancer cells grow excessively and continuously, invading surrounding tissue and metastasize to distal organs leading to death. Despite the attack of an immune mechanism or anticancer therapy, cancer cells survive, continually develop, and cell groups that are most suitable for survival selectively propagate. Cancer cells are living bodies with a high degree of viability, which occur by the mutation of a large number of genes. In order that one cell is converted to a cancer cell and developed to a malignant cancer lump that is detectable in clinics, the mutation of a large number of genes must occur. Thus, in order to diagnose and treat cancer at the root, approaches at a gene level are necessary.

Recently, genetic analysis is actively being attempted to diagnose cancer. The simplest typical method is to detect the presence of ABL:BCR fusion genes (the genetic characteristic of leukemia) in blood by PCR. The method has an accuracy rate of more than 95%, and after the diagnosis and therapy of chronic myelocytic leukemia using this simple and easy genetic analysis, this method is being used for the assessment of the result and follow-up study. However, this method has the deficiency that it can be applied only to some blood cancers.

Recently, genetic testing using a DNA in serum or plasma is actively being attempted. This is a method of detecting a cancer-related gene that is isolated from cancer cells and released into blood and present in the form of a free DNA in serum. It is found that the concentration of DNA in serum is increased by a factor of 5-10 times in actual cancer patients as compared to that of normal persons, and such increased DNA is released mostly from cancer cells. The analysis of cancer-specific gene abnormalities, such as the mutation, deletion and functional loss of oncogenes and tumor-suppressor genes, using such DNAs isolated from cancer cells, allows the diagnosis of cancer. In this effort, there has been an active attempt to diagnose lung cancer, head and neck cancer, breast cancer, colon cancer, and liver cancer by examining the promoter methylation of mutated K-Ras oncogenes, p53 tumor-suppressor genes and p16 genes in serum, and the labeling and instability of microsatellite (Chen, X. Q. et al., Clin. Cancer Res., 5:2297, 1999; Esteller, M. et al., Cancer Res., 59:67, 1999; Sanchez-Cespedes, M. et al., Cancer Res., 60:892, 2000; Sozzi, G. et al., Clin. Cancer Res., 5:2689, 1999).

In samples other than blood, the DNA of cancer cells can also be detected. A method is being attempted in which the presence of cancer cells or oncogenes in sputum or bronchoalveolar lavage of lung cancer patients is detected by a gene or antibody test (Palmisano, W. A. et al., Cancer Res., 60:5954, 2000; Sueoka, E. et al, Cancer Res., 59:1404, 1999). Additionally, other methods of detecting the presence of oncogenes in feces of colon and rectal cancer patients (Ahlquist, D. A. et al., Gastroenterol., 119:1219, 2000) and detecting promoter methylation abnormalities in urine and prostate fluid (Goessl, C. et al., Cancer Res., 60:5941, 2000) are being attempted. However, in order to accurately diagnose cancers that cause a large number of gene abnormalities and show various mutations characteristic of each cancer, a method, by which a large number of genes are simultaneously analyzed in an accurate and automatic manner, is required. However, such a method is not yet established.

Accordingly, methods of diagnosing cancer by the measurement of DNA methylation are being proposed. When the promoter CpG island of a certain gene is hyper-methylated, the expression of such a gene is silenced. This is interpreted to be a main mechanism by which the function of this gene is lost even when there is no mutation in the protein-coding sequence of the gene in a living body. Also, this is analyzed as a factor by which the function of a number of tumor-suppressor genes in human cancer is lost. Thus, detecting the methylation of the promoter CpG island of tumor-suppressor genes is greatly needed for the study of cancer. Recently, an attempt has actively been conducted to determine promoter methylation, by methods such as methylation-specific PCR (hereinafter, referred to as MSP) or automatic DNA sequencing, for diagnosis and screening of cancer.

A significant number of diseases are caused by genetic abnormalities, and the most frequent forms of genetic abnormalities are changes in gene-coding sequences. Such genetic changes are called mutations. When there are mutations in any gene, the structure and function of a protein coded by such a gene are changed, and hindrance and deletion are caused, and such a mutated protein causes a disease. However, even if there are no mutations in a certain gene, an abnormality in the expression of this gene can cause disease. A typical example is methylation where methyl groups are attached to gene transcriptional regulatory sites, i.g., the cytosine base sites of CpG islands, in which case the expression of this gene is blocked. This is called an epigenetic change, which is transferred to offspring cells in a similar manner to mutations, and causes the same effect, i.e., the loss of expression of the corresponding protein. The most typical change is that the expression of tumor-suppressor genes is blocked by the methylation of promoter CpG islands in cancer cells, and this blocked expression an important mechanism of causing cancer (Robertson, K. D. & Jones, P. A., Carcinogensis, 21:461, 2000).

For the accurate diagnosis of cancer, it is important to detect not only a mutated gene but also to determine a mechanism, where the mutation of this gene appears. While previous studies have been conducted by focusing on the mutations of a coding sequence, i.e., micro-changes, such as point mutations, deletions and insertions, or macroscopic chromosomal abnormalities, recently, epigenetic changes are reported to be as important as these mutations, and a typical example of such epigenetic changes is the methylation of promoter CpG islands.

In the genomic DNA of mammal cells, there is the fifth base in addition to A, C, G and T, namely, 5-methylcytosine, in which a methyl group is attached to the fifth carbon of the cytosine ring (5-mC). 5-mC is always attached only to the C of a CG dinucleotide (5′-mCG-3′), which is frequently marked CpG. The C of CpG is mostly methylated by attachment with a methyl group. The methylation of this CpG inhibits a repetitive sequence in genomes, such as alu or transposon, from being expressed. Also, this CpG is a site where an epigenetic change in mammalian cells appears most often. The 5-mC of this CpG is naturally deaminated to T, and thus, the CpG in mammal genomes shows only 1% of frequency, which is much lower than a normal frequency (¼×¼=6.25%).

Regions that CpG is exceptionally integrated are known as CpG islands. The CpG islands refer to sites which are 0.2-3 kb in length, and have a C+G content of more than 50% and a CpG ratio of more than 3.75%. There are about 45,000 CpG islands in the human genome, and they are mostly found in promoter regions regulating the expression of genes. Actually, the CpG islands occur in the promoters of housekeeping genes accounting for about 50% of human genes (Cross, S. H. & Bird, A. P., Curr. Opin. Gene Develop., 5:309, 1995).

In the somatic cells of normal persons, the CpG islands of such housekeeping gene promoter sites are un-methylated, but imprinted genes and the genes on inactivated X chromosomes are methylated such that they are not expressed during development.

During a cancer-causing process, methylation is found in promoter CpG islands, and the restriction on the corresponding gene expression occurs. Particularly, if methylation occurs in the promoter CpG islands of tumor-suppressor genes that regulate cell cycle or apoptosis, restore DNA, are involved in the adhesion of cells and the interaction between cells, and/or suppress cell invasion and metastasis, such methylation blocks the expression and function of such genes in the same manner as the mutations of a coding sequence, thereby promoting the development and progression of cancer. In addition, partial methylation also occurs in the CpG islands according to aging.

An interesting fact is that, in the case of genes whose mutations are attributed to the development of cancer in congenital cancer but do not occur in acquired cancer, the methylation of promoter CpG islands occurs instead of mutation. Typical examples include the promoter methylation of genes, such as acquired renal cancer VHL (von Hippel Lindau), breast cancer BRCA1, colon cancer MLH1, and stomach cancer E-CAD. In addition, in about half of all cancers, the promoter methylation of p16 or the mutation of Rb occurs, and the remaining cancers show the mutation of p53 or the promoter methylation of p73, p14 and the like.

An important fact is that an epigenetic change caused by promoter methylation causes a genetic change (i.e., the mutation of a coding sequence), and the development of cancer is progressed by the combination of such genetic and epigenetic changes. In a MLH1 gene as an example, there is the circumstance in which the function of one allele of the MLH1 gene in colon cancer cells is lost due to its mutation or deletion, and the remaining one allele does not function due to promoter methylation. In addition, if the function of MLH1, which is a DNA restoring gene, is lost due to promoter methylation, the occurrence of mutation in other important genes is facilitated to promote the development of cancer.

Most cancers show three common characteristics with respect to CpG, namely, hypermethylation of the promoter CpG islands of tumor-suppressor genes, hypomethylation of the remaining CpG base sites, and an increase in the activity of methylation enzyme, namely, DNA cytosine methyltransferase (DNMT) (Singal, R. & Ginder, G. D., Blood, 93:4059, 1999; Robertson, K. & Jones, P. A., Carcinogensis, 21:461, 2000; Malik, K. & Brown, K. W., Brit. J. Cancer, 83:1583, 2000).

When promoter CpG islands are methylated, the reason why the expression of the corresponding genes is blocked is not clearly established, but is presumed to be because a methyl CpG-binding protein (MECP) or a methyl CpG-binding domain protein (MBD), and histone deacetylase, bind to methylated cytosine thereby causing a change in the chromatin structure of chromosomes and a change in histone protein.

There are dispute about whether the methylation of promoter CpG islands directly causes the development of cancer or is a secondary change after the development of cancer. However, it is clear that the promoter methylation of tumor-related genes is an important index to cancer, and thus, can be used in many applications, including the diagnosis and early detection of cancer, the prediction of the risk of the development of cancer, the prognosis of cancer, follow-up examination after treatment, and the prediction of a response to anticancer therapy. Recently, an attempt to examine the promoter methylation of tumor-related genes in blood, sputum, saliva, feces or urine and to use the examined results for the diagnosis and treatment of various cancers, has been actively conducted (Esteller, M. et al., Cancer Res., 59:67, 1999; Sanchez-Cespedez, M. et al., Cancer Res., 60:892, 2000; Ahlquist, D. A. et al., Gastroenterol., 119:1219, 2000).

In order to maximize the accuracy of cancer diagnosis using promoter methylation, analyze the development of cancer according to each stage and discriminate a change according to cancer and aging, an examination that can accurately analyze the methylation of all the cytosine bases of promoter CpG islands is required. Currently, a standard method for this examination is a bisulfite genome-sequencing method, in which a sample DNA is treated with sodium bisulfite, and all regions of the CpG islands of a target gene to be examined is amplified by PCR, and then, the base sequence of the amplified regions is analyzed. However, this examination has the problem that there are limitations of the number of genes or samples that can be examined at a given time. Other problems are that automation is difficult, and much time and expense are required.

The methylation of promoter CpG islands has a deep connection with physiological phenomena, such as the development and differentiation of the human body, and also aging, the development of various cancers and diseases. Particularly, the methylation of the promoter CpG islands of tumor-related genes can act as an index of cancer since they play an important role in the development and progression of cancer. In particular, in cervical cancer, for instance, stages of cancer progression have been categorized, such as “SIL (squamous intraepithelial lesion)”, which indicates dysplasia generally; “LSIL”, which indicates mild dysplasia; “HSIL”, which indicates moderate to severe dysplasia; “CIS (carcinoma in situ); and Squamous cell carcinoma. Accordingly, it is desirable to find marker genes that are specific for these stages of cancer progression.

However, conventional methods utilize amplification of regions of genes containing CpG island by methylation specific PCR (MSP) together with a base sequence analysis method (bisulfite genome-sequencing method). Furthermore, there is no method that can analyze various changes of the promoter methylation of many genes at a given time in an accurate, rapid and automatic manner, and can be applied to the diagnosis, early diagnosis or assessment of each stage of various cancers in clinical practice.

In the area of screening of new tumor suppressor genes associated with methylation, many studies have been performed. Examples of the existing screening methods include: a method where the genomic DNAs of cancer tissues and normal tissues are restricted with methylation-related restriction enzymes, and many DNA fragments obtained are all cloned, and then DNA fragments having the difference between cancer tissues and normal tissues are selected, sequenced and screened; and a method using a binding column that recognizes CpG islands (Huang, T. H. et al., Hum. Mol. Genet., 8:459, 1999; Cross, S. H. et al., Nat. Genet., 6:236, 1994). However, such methods have shortcomings in that they require much time, and are not efficient to screen gene candidates and also are difficult to apply in actual clinical practice.

Accordingly, the present invention is directed to screening for methylated promoter markers involved in cell conversion especially cancer cell conversion and treatment of cancer.

### SUMMARY OF THE INVENTION

The present invention is directed to a systematic approach to identifying methylation regulated marker genes in cell conversion. In one aspect of the invention, (1) the genomic expression content between a converted and unconverted cell or cell line is compared and a profile of the expressed genes that are more abundant in the unconverted cell or cell line is categorized; (2) a converted cell or cell line is treated with a methylation inhibitor, and genomic expression content between the methylation inhibitor treated converted cell or cell line and untreated converted cell or cell line is compared and a profile of the more abundantly expressed genes in the methylation inhibitor treated converted cell or cell line is categorized; (3) profiles of genes from those obtained in (1) and (2) above are compared and the genes that appear in both groups are considered to be candidate methylation regulated marker genes in converting a cell from the unconverted state to the converted form. Further confirmation may be needed such as by examining the sequence of the gene to determine if there is a CpG sequence present, and by carrying out further biochemical assays to determine whether the genes are actually methylated.

The present invention is also based on the finding that by using this system several genes are identified as being differentially methylated in cervical cancer as well as at various dysplasic stages of the tissue in the progression to cervical cancer. This discovery is useful for cervical cancer screening, risk-assessment, prognosis, disease identification, disease staging and identification of therapeutic targets. The identification of genes that are methylated in cervical cancer and its various grades of lesion allows for the development of accurate and effective early diagnostic assays, methylation profiling using multiple genes, and identification of new targets for therapeutic intervention. Further, the methylation data may be combined with other non-methylation related biomarker detection methods to obtain a more accurate diagnostic system for cervical cancer.

In one embodiment, the invention provides a method of diagnosing various stages or grades of cervical cancer progression comprising determining the state of methylation of one or more nucleic acid biomarkers isolated from the subject as described above. The state of methylation of one or more nucleic acids compared with the state of methylation of one or more nucleic acids from a subject not having the cellular proliferative disorder of cervical tissue is indicative of a certain stage of cervical disorder in the subject. In one aspect of this embodiment, the state of methylation is hypermethylation.

In one aspect of the invention, nucleic acids are methylated in the regulatory regions. In another aspect, since methylation begins from the outer boundaries of the regulatory region and working inward, detecting methylation at the outer boundaries of the regulatory region allows for early detection of the gene involved in cell conversion such as cancer.

In one aspect, the invention provides a method of diagnosing a cellular proliferative disorder of cervical tissue in a subject by detecting the state of methylation of one or more of the following exemplified nucleic acids: Nucleoporin 98 kDa, Selenoprotein X, 1, DKFZP4340047 protein, Zinc finger protein 324, Testis-specific kinase 2, Corin, serine protease, GLI-Kruppel family member GLI2, Spermidine/spermine N1-acetyltransferase, Scaffold attachement factor B, Leucine-rich repeats and calponin homology (CH) domain containing 4, Laminin, beta 2 (Laminin S), ATPase Na+/K+ transporting, beta 2 polypeptide, Tubulin, beta polypeptide, Aldehyde dehydrogenase 3 family, member B1, Leukocyte tyrosine kinase, Procollagen C endopeptiase enhancer, Protein tyrosine phosphatase, receptor type, U, TAF10 RNA polymerase II, TATA box binding protein (TBP)-associated factor 30 kDa, Fibroblast growth factor receptor 1 (fms-related tyrosine kinase 2, Pfeiffer syndrome), and DNA-damage-inducible transcript 3, and combinations thereof.

In another embodiment of the invention, the invention provides a method of diagnosing a high grade lesion of cellular proliferative disorder of cervical tissue in a subject by detecting the state of methylation of one or more of the following exemplified nucleic acids: ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin A1; CCND2 (NT_{—}009759): Cyclin D2; EPHA5 (NT_{—}022778): EphA5; HOXA1 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin, and combinations thereof.

Another embodiment of the invention provides a method of determining a predisposition to a cellular proliferative disorder of cervical tissue in a subject. The method includes determining the state of methylation of one or more nucleic acids isolated from the subject, wherein the state of methylation of one or more nucleic acids compared with the state of methylation of the nucleic acid from a subject not having a predisposition to the cellular proliferative disorder of cervical tissue is indicative of a cell proliferative disorder of cervical tissue in the subject. Some of the exemplified nucleic acids can be nucleic acids encoding Nucleoporin 98 kDa, Selenoprotein X, 1, DKFZP4340047 protein, Zinc finger protein 324, Testis-specific kinase 2, Corin, serine protease, GLI-Kruppel family member GLI2, Spermidine/spermine N1-acetyltransferase, Scaffold attachement factor B, Leucine-rich repeats and calponin homology (CH) domain containing 4, Laminin, beta 2 (Laminin S), ATPase Na+/K+ transporting, beta 2 polypeptide, Tubulin, beta polypeptide, Aldehyde dehydrogenase 3 family, member B1, Leukocyte tyrosine kinase, Procollagen C endopeptiase enhancer, Protein tyrosine phosphatase, receptor type, U, TAF10 RNA polymerase II, TATA box binding protein (TBP)-associated factor 30 kDa, Fibroblast growth factor receptor 1 (fins-related tyrosine kinase 2, Pfeiffer syndrome), and DNA-damage-inducible transcript 3, ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin A1; CCND2 (NT_{—}009759): Cyclin D2; EPHA5 (NT_{—}022778): EphA5; HOXA11 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT_{—}086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin, and combinations thereof.

Still another embodiment of the invention provides a method for detecting a cellular proliferative disorder of cervical tissue in a subject. The method includes contacting a specimen containing at least one nucleic acid from the subject with an agent that provides a determination of the methylation state of at least one nucleic acid. The method further includes identifying the methylation states of at least one region of at least one nucleic acid, wherein the methylation state of the nucleic acid is different from the methylation state of the same region of nucleic acid in a subject not having the cellular proliferative disorder of cervical tissue.

Yet a further embodiment of the invention provides a kit useful for the detection of a cellular proliferative disorder in a subject comprising carrier means compartmentalized to receive a sample therein; and one or more containers comprising a first container containing a reagent that sensitively cleaves unmethylated nucleic acid and a second container containing target-specific primers for amplification of the biomarker.

In another embodiment, the invention is directed to a method for discovering a methylation marker gene for the conversion of a cell comprising: (i) comparing converted and unconverted cell gene expression content to identify a gene that is present in greater abundance in the unconverted cell; (ii) treating a converted cell with a demethylating agent and comparing its gene expression content with gene expression content of an untreated converted cell to identify a gene that is present in greater abundance in the cell treated with the demethylating agent; and (iii) identifying a gene that is common to the identified genes in steps (i) and (ii), wherein the common identified gene is the methylation marker gene.

The method may comprise reviewing the sequence of the identified gene and discarding the gene for which the promoter sequence does not have a CpG island. The comparing may be carried out by direct comparison or indirect comparison. The converted cell may be cancer cell or blood cell. The cancer may be melanoma, carcinoma, or sarcoma, preferably, cervical cancer. The converted cell may represent cervical dysplasia. The dysplasia may include squamous intraepithelial lesion (SIL), low squamous intraepithelial lesion (LSIL), high squamous intraepithelial lesion (HSIL), carcinoma in situ (CIS) or cancer.

The method above may further comprise confirming the methylation marker gene, which comprises assaying for methylation of the common identified gene in the converted cell, wherein the presence of methylation in the promoter region of the common identified gene confirms that the identified gene is the marker gene.

The assay for methylation of the identified gene may be carried out by (i) identifying primers that span a methylation site within the nucleic acid region to be amplified, (ii) treating the genome of the converted cell with a methylation specific restriction endonuclease, and (iii) amplifying the nucleic acid by contacting the genomic nucleic acid with the primers, wherein successful amplification indicates that the identified gene is methylated, and unsuccessful amplification indicates that the identified gene is not methylated.

The converted cell genome may be treated with an isoschizomer of the methylation sensitive restriction endonuclease that cleaves both methylated and unmethylated CpG-sites as a control. Detecting the presence of amplified nucleic acid may be carried out by hybridization with a probe. The probe may be immobilized on a solid substrate. The amplification may be carried out by PCR, real time PCR, or amplification or linear amplification using isothermal enzyme. Detection of methylation on the outer part of the promoter may be indicative of early detection of cell conversion.

The invention is also directed to a method of identifying a converted cell comprising assaying for the methylation of the marker gene identified in the method described above. The invention is also directed to a method of diagnosing cancer or a stage in the progression of the cancer in a subject comprising assaying for the methylation of the marker gene identified using the method described above. In this method, the cancer may be cervical cancer. The dysplasia may be observed in sample taken from scrape, biopsy, blood or urine.

These and other objects of the invention will be more fully understood from the following description of the invention, the referenced drawings attached hereto and the claims appended hereto.

### BRIEF DESCRIPTION OF THE DRAWINGS

The present invention will become more fully understood from the detailed description given herein below, and the accompanying drawings which are given by way of illustration only, and thus are not limitative of the present invention, and wherein;

FIG. 1 shows a schematic diagram for systematic biomarker discovery.

FIG. 2 shows a schematic diagram for a systematic method for discovering cervical cancer biomarker.

FIG. 3 shows a flowchart for cervical cancer biomarker discovery.

FIG. 4 shows a schematic diagram to conduct methylation assay by enzyme digestion and subsequent gene amplification analysis to determine whether a candidate marker gene is actually methylated.

FIG. 5 shows gene expression profile of 20 promoter methylated genes in normal, non-tumorous, and tumorous cervical tissue. These genes were identified based on the genes that were down regulated in cervical tumor cells.

FIG. 6 shows gene methylation status of the 20 identified genes at various stages of cancer progression, including Normal (Pap I), Pap II including ASCUS, LSIL, HSIL, CIS, and cancer. Gene expression was determined using cervical scrape.

FIG. 7 shows gene methylation status of the 20 identified genes at various stages of cancer progression, including Normal (Pap I), LSIL, HSIL, CIS, and cancer. Gene expression was determined using biopsy sample.

FIG. 8 shows discovery strategy for methylation markers for cervical high grade lesion. High grade lesion is defined as cervical tissue dysplasia that includes HSIL, CIS and cancer.

FIG. 9 shows a flowchart for cervical high grade lesion biomarker discovery.

FIG. 10 shows gene expression profiles of 28 identified genes at various stages of cancer progression, including Normal (Pap I), LSIL, HSIL, CIS, and cancer. Gene expression was determined using biopsy samples.

FIG. 11 shows gene methylation status of the 28 identified genes at various stages of cancer progression, including Normal (Pap I), Pap II, LSIL, HSIL, CIS, and cancer. Gene expression was determined using biopsy samples.

FIG. 12 shows gene methylation status of 22 consolidated identified genes at various stages of cancer progression, including Normal (Pap I), Pap II, ASCUS, LSIL, HSIL, CIS, and cancer. Gene expression was determined using cervical scrapes.

### DETAILED DESCRIPTION OF THE PREFERRED EMBODIMENTS

In the present application, “a” and “an” are used to refer to both single and a plurality of objects.

As used herein, “cell conversion” refers to the change in characteristics of a cell from one form to another such as from normal to abnormal, non-tumorous to tumorous, undifferentiated to differentiated, stem cell to non-stem cell. Further, the conversion may be recognized by morphology of the cell, phenotype of the cell, biochemical characteristics and so on. There are many examples, but a few examples may include normal cell converting to tumor cell or a stem cell converting to a neuron and so on. Moreover, such conversion may include tissue conversion. For instance, cervical tissue dysplasia is manifest by the presence of abnormal cells. Markers for such tissue conversion are within the purview of cell conversion.

Still further, conversion also includes cancer. Types of cancer may include without limitation carcinoma, melanoma and sarcoma. Subtypes of cancer may include without limitation bladder carcinoma, brain tumor, breast cancer, cervical cancer, colorectal cancer, esophageal cancer, endometrial cancer, hepatocellular carcinoma, gastrointestinal stromal tumor (GIST), laryngeal cancer, lung cancer, osteosarcoma, ovarian ancer, pancreatic cancer prostate cancer, renal cell carcinoma or thyroid cancer.

As used herein, “demethylating agent” refers to any agent, including but not limited to chemical or enzyme, that either removes a methyl group from the nucleic acid or prevents methylation from occurring. Examples of such demethylating agents include without limitation nucleotide analogs such as 5-azacytidine, 5 aza 2′-deoxycytidine (DAC), arabinofuranosyl-5-azacytosine, 5-fluoro-2′-deoxycytidine, pyrimidone, trifluoromethyldeoxycytidine, pseudoisocytidine, dihydro-5-azacytidine, AdoMet/AdoHcy analogs as competitive inhibitors such as AdoHcy, sinefungin and analogs, 5′deoxy-5′-S-isobutyladenosine (SIBA), 5′-methylthio-5′deoxyadenosine (MTA), drugs influencing the level of AdoMet such as ethionine analogs, methionine, L-cis-AMB, cycloleucine, antifolates, methotrexate, drugs influencing the level of AdoHcy, dc-AdoMet and MTA such as inhibitors of AdoHcy hydrolase, 3-deaza-adenosine, neplanocin A, 3-deazaneplanocin, 4′-thioadenosine, 3-deaza-aristeromycin, inhibitors of ornithine decarboxylase, α-difluoromethylornithine (DFMO), inhibitors of spermine and spermidine synthetase, S-methyl-5′-methylthioadenosine (MTA), L-cis-AMB, AdoDATO, MGBG, inhibitors of methylthioadenosine phosphorylase, difluoromethylthioadenosine (DFMTA), other inhibitors such as methinin, spermine/spermidine, sodium butyrate, procainamide, hydralazine, dimethylsulfoxide, free radical DNA adducts, UV-light, 8-hydroxy guanine, N-methyl-N-nitrosourea, novobiocine, phenobarbital, benzo[a]pyrene, ethylmethansulfonate, ethylnitrosourea, N-ethyl-N′-nitro-N-nitrosoguanidine, 9-aminoacridine, nitrogen mustard, N-methyl-N′-nitro-N-nitrosoguanidine, diethylnitrosamine, chlordane, N-acetoxy-N-2-acetylaminofluorene, aflatoxin B1, nalidixic acid, N-2-fluorenylacetamine, 3-methyl-4′-(dimethylamino)azobenzene, 1,3-bis(2-chlorethyl)-1-nitrosourea, cyclophosphamide, 6-mercaptopurine, 4-nitroquinoline-1-oxide, N-nitrosodiethylamine, hexamethylenebisacetamide, retinoic acid, retinoic acid with cAMP, aromatic hydrocarbon carcinogens, dibutyryl cAMP, or antisense mRNA to the methyltransferase (Zingg et al., Carcinogenesis, 18:5, pp. 869-882, 1997). The contents of this reference is incorporated by reference in its entirety especially with regard to the discussion of methylation of the genome and inhibitors thereof.

As used herein, “direct comparison” refers to a competitive binding to a probe among differentially labeled nucleic acids from more than one source in order to determine the relative abundance of one type of differentially labeled nucleic acid over the other.

As used herein, “cervical dysplasia” refers to the appearance of abnormal cells on the surface of the cervix. These changes in cervical tissue are classified as mild, moderate, or severe. While dysplasia itself does not cause health problems, it is considered to be a precancerous condition. Left untreated, dysplasia sometimes progresses to an early form of cancer known as cervical carcinoma in situ, and eventually to invasive cervical cancer. Mild dysplasia is the most common form, and up to 70% of these cases regress on their own (i.e., the cervical tissue returns to normal without treatment). Moderate and severe dysplasia are less likely to self-resolve and have a higher rate of progression to cancer. The greater the abnormality, the higher the risk for developing cervical cancer. Detecting and treating dysplasia early is essential to prevent cancer.

As used herein, “hypermethylation” refers to the methylation of a CpG island.

As used herein, “high or higher grade lesion” refers to moderate or severe dysplasia. In the present invention, it is meant to include any cell cytology that is at least as severe as high squamous intraepithelial lesion (HSIL), including without limitation HSIL, carcinoma in situ (CIS) or cancer.

As used herein, “indirect comparison” refers to assessing the level of nucleic acid from a first source with the level of the same allelelic nucleic acid from a second source by utilizing a reference probe to which is separately hybridized the nucleic acid from the first and second sources and the results are compared to determine the relative amounts of the nucleic acids present in the sample without direct competitive binding to the reference probe.

As used herein, “low or lower grade lesion” refers to normal cells or mild dysplasia. In the present invention, it is meant to include any cell cytology that is at least as low in severity as squamous intraepithelial lesion (LSIL), including without limitation LSIL or normal cells.

Screening for Methylation Regulated Biomarkers

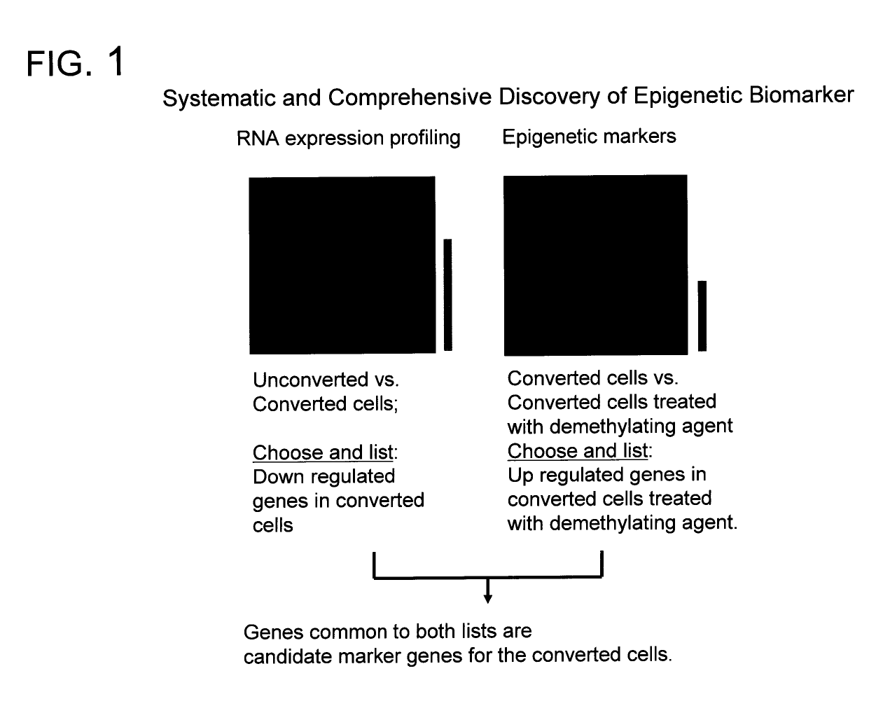

The present invention is directed to a method of determining biomarker genes that are methylated when the cell or tissue is converted or changed from one type of cell to another. As used herein, “converted” cell refers to the change in characteristics of a cell or tissue from one form to another such as from normal to abnormal, non-tumorous to tumorous, undifferentiated to differentiated and so on. See FIG. 1.

Thus, the present invention is directed to a systematic approach to identifying methylation regulated marker genes in cell conversion. In one aspect of the invention, (1) the genomic expression content between a converted and unconverted cell or cell line is compared and a profile of the more abundantly expressed genes in the unconverted cell or cell line is categorized; (2) a converted cell or cell line is treated with a methylation inhibitor, and genomic expression content between the methylation inhibitor treated converted cell or cell line and untreated converted cell or cell line is compared and a profile of the more abundantly expressed genes in the methylation inhibitor treated converted cell or cell line is categorized; (3) profiles of genes from those obtained in (1) and (2) above are compared and overlapping genes are considered to be methylation regulated marker genes in converting a cell from the unconverted state to the converted form.

It should be added also in step (2) above, the converted cell line may include a blood cell line.

In addition to the above, in order to further fine-tune the list of candidate biomarkers and also to determine whether the candidate biomarkers so obtained above are indeed methylated under conversion conditions, a nucleic acid methylation detecting assay is carried out. Any number of numerous ways of detecting methylation on a DNA fragment may be used. By way of example only and without limitation, one such way is as follows. Genomic DNA is treated with a methylation sensitive restriction enzyme, and probed with marker specific gene sequence directed to the methylation region. Detection of an uncleaved probed region indicates that methylation has occurred at the probed site.

One way to practice the invention is by utilizing microarray technology as follows:

(1) Converted cell expression library and non-converted cell expression library are differentially labeled with preferably fluorescent labels, Cy3 which produces green color, and Cy5 which emanates red color. They are competitively bound to a microarray immobilized with a set of known gene probes. The genes that are differentially more expressed in the unconverted cells are identified. Alternatively, an indirect comparison method may be used.

(2) Converted cell line is treated with a demethylating agent and the expression library is labeled with a fluorescent label. A differentially labeled expression library from a converted cell line that has not been treated with the demethylating agent is also obtained. The two libraries are competitively bound on a microarray substrate immobilized with a set of known gene probes. The genes that are differentially more expressed in the converted cells treated with the demethylating agent are identified. These genes are presumably reactivated under demethylating conditions. Alternatively, an indirect comparison method may be used.

(3) The identified genes from the two sets of experiments above are compared and genes common to both lists are chosen.

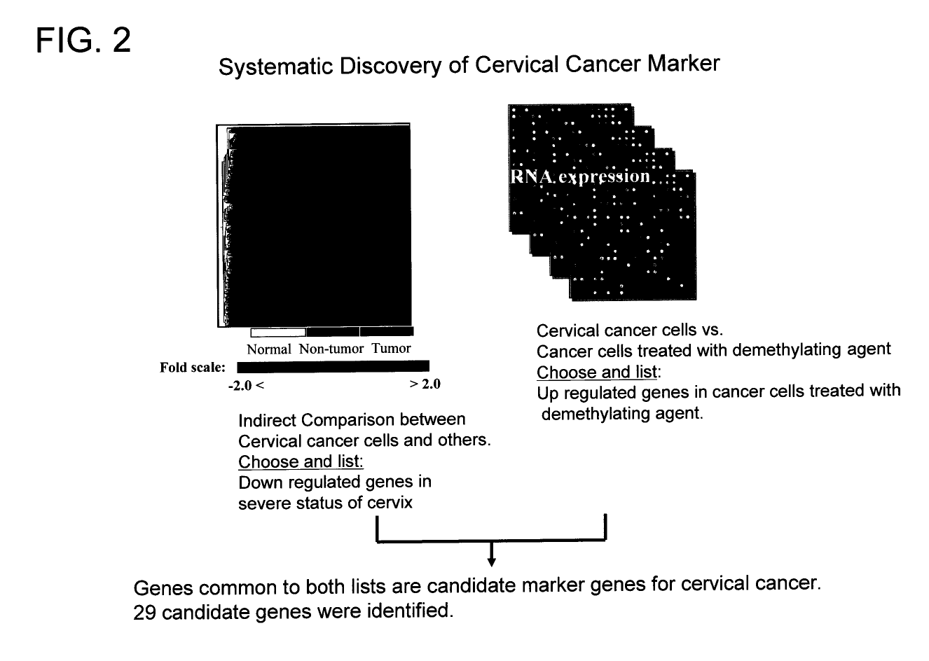

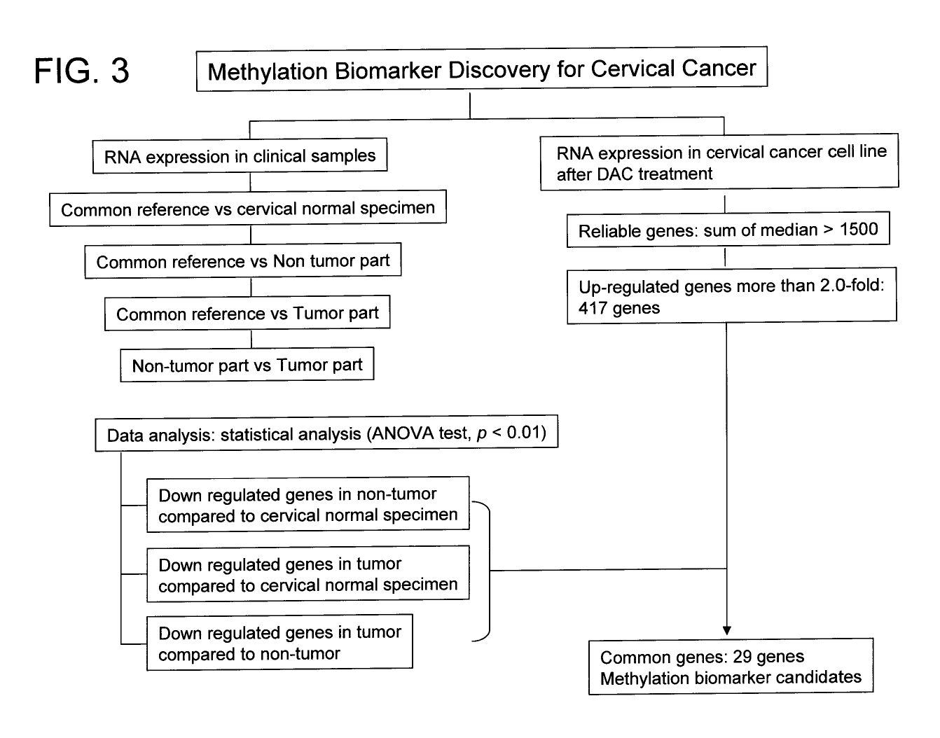

Again, it is understood that such comparison in gene expression between the converted and unconverted cells and between cells treated with demethylating agent and not treated with demethylating agent may be carried out by direct competitive binding to a set of probes. Alternatively, the comparison may be indirect. For instance, the expressed genes may be bound to a set of known reference gene probes each separately. Thus, the relative abundance of expressed genes from the various cells can be compared indirectly. The set of reference gene probes are generally optimized so that they contain as complete a set of expressed genes as possible. See FIGS. 2 and 3.

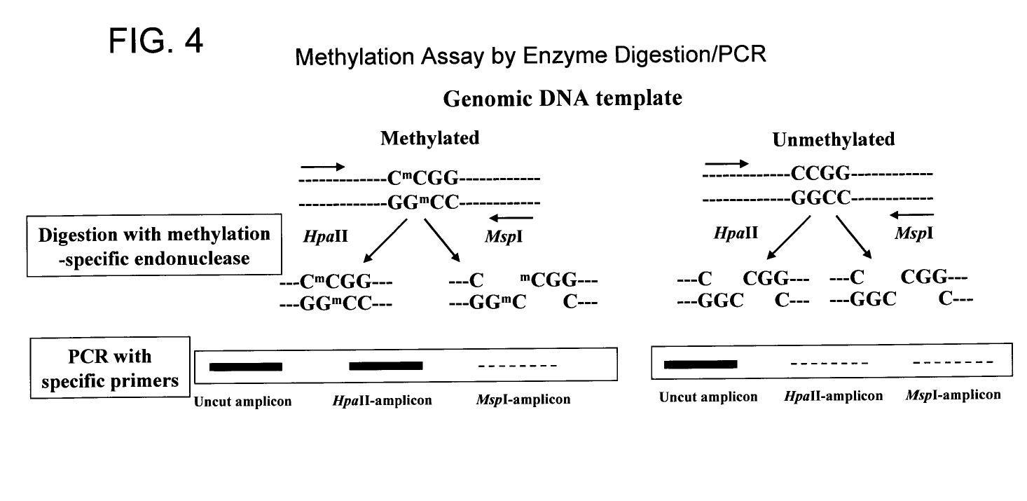

(4) The nucleic acid sequence of the promoter regions of the genes are examined to determine whether there are CpG islands within them. Genes with promoters that do not possess CpG islands are discarded. The remaining genes are assayed for their level of methylation. This can be accomplished using a variety of means. In one embodiment, the genome from converted cells is digested with methylation sensitive restriction endonuclease. Nucleic acid amplification is carried out using various primers wherein the methylation site is located within the region to be amplified. When the nucleic acid amplification step is carried out, successful amplification indicates that methylation has occurred because the gene was not cleaved by the methylation sensitive restriction endonuclease. The absence of an amplified product indicates that methylation did not occur because the gene was digested by the methylation sensitive restriction endonuclease. Results of such experiments are shown in FIG. 4.

Cervical Cancer Biomarkers

An exemplary use of the inventive systematic approach to biomarker discovery is in cancer detection. In particular and by way of example only, biomarkers for cervical cancer detection is provided in the present application. Further, biomarkers in each cytological stage of development of cervical cancer is also provided.

Cytology

The cytology of cancer cells differs significantly from normal cells, and physicians use the unique cellular features seen on biopsy samples to determine the diagnosis and assess the prognosis of a cancer.

Briefly, the Bethesda system of cervical cytology nomenclature system includes “Normal”, which indicates normal; “Infection” and “Reactive/Reparative”, which indicate inflammatory atypia; “ASC-US/ASC-H”, which indicates squamous atypia/HPV atypia; “SIL (squamous intraepithelial lesion)”, which indicates dysplasia generally; “LSIL”, which indicates mild dysplasia; “HSIL”, which indicates moderate to severe dysplasia; “CIS (carcinoma in situ); and Squamous cell carcinoma.

The criteria for diagnosing precancerous lesions of the cervix vary somewhat among doctors, but important characteristics include cellular immaturity, cellular disorganization, nuclear abnormalities, and increased mitotic activity. Some of the cellular abnormalities seen in cervical cancer include the following:

(i) Carcinoma in situ is diagnosed when normal endocervical gland cells are replaced by tall, irregular columnar cells that have stratified nuclei and increased cell division.

(ii) Squamous carcinomas have small to medium-sized nuclei and abundant cytoplasm.

(iii) Adenocarcinomas usually present with a wide variety of cell types, growth patterns, and degrees of differentiation.

The Pap test is used to identify the presence of abnormal cell growth that could develop into—or already is—cancerous. Most laboratories in the United States now use the Bethesda System to report Pap test results. The Bethesda System uses descriptive terms rather than class numbers, which were used to report Pap test results in the past.

The Bethesda System divides cervical cell abnormalities into three major categories:

(i) ASCUS—atypical squamous cells of undetermined significance. Squamous cells are the thin flat cells that form the surface of the cervix.

(ii) LSIL—low-grade squamous intraepithelial lesion. Low-grade means there are early changes in the size and shape of cells. The word lesion refers to an area of abnormal tissue; intraepithelial means that the abnormal cells are present only in the surface layer of cells.

(iii) HSIL—high-grade squamous intraepithelial lesion. High-grade means that there are more marked changes in the size and shape of the abnormal (precancerous) cells that look very different from normal cells.

ASCUS and LSIL are considered mild abnormalities. HSIL is more severe and has a higher likelihood of progressing to invasive cancer.

The classes of the Pap System are as follows:

(i) Class I—normal

(ii) Class II—squamous cell abnormalities, infection, reactive changes

(iii) Class IIR—atypical squamous cells of undetermined significance, HPV presence

(iv) Class III—mild, moderate, severe dysplasia

(v) Class IV—carcinoma in situ

(vi) Class V—invasive squamous carcinoma.

Cervical Cancer Biomarker—Experiment I: Using Cancer Tumor Cells for Comparison with Normal Cells

In practicing the invention, it is understood that “normal” cells are those that do not show any abnormal morphological or cytological changes. “Tumor” cells are cancer cells. “Non-tumor” cells are those cells that were part of the diseased tissue but were not considered to be the tumor portion.

Cervical tumor cell gene expression content was indirectly compared between normal cell and tumor cell gene expression content in a microarray competitive hybridization format. A common reference was competed with normal cell gene content; common reference vs. non-tumor gene content; and common reference vs. tumor. Genes that were repressed in non-tumor and tumor cells as compared with normal cells were found and noted. And further, of these genes, the genes that were suppressed in tumor cells compared with non-tumor cells were further noted and listed and considered as the tumor suppressed genes.

Alternatively, the gene expression content from tumor may be directly competed with non-tumor and/or normal cells in a microarray hybridization format to obtain the tumor suppressed genes.

Separately, a cervical cancer cell line C33A was treated with a demethylating agent DAC and assayed for reactivation of genes that are normally repressed in tumor cells. Overlapping genes between the tumor suppressed gene set and the demethylation reactivated gene set were considered to be candidate genes for cervical cancer biomarkers. Twenty nine (29) such overlapping genes were found. These genes were then analyzed in silico to determine whether they contained the requisite CpG island motif. A few genes (5 genes) did not contain them and were removed. Further biochemical testing of the remaining 24 genes was needed to determine whether the candidate genes were actually methylated when isolated from tumor cells. Methylation sensitive enzyme/nucleic acid sequence based amplification (NASBA) analysis such as Hpa II/MspI enzyme digestion/PCR (or enzyme digestion post-PCR) further removed a few other genes (4 genes) that were not methylated in any of the four cervical cancer cell lines (C33A, SiHa, HeLa and Caski). To further confirm biochemically that the candidate gene was indeed methylated in tumor cells, bisulfite sequencing assays were conducted and methylation of the final 20 genes was verified.

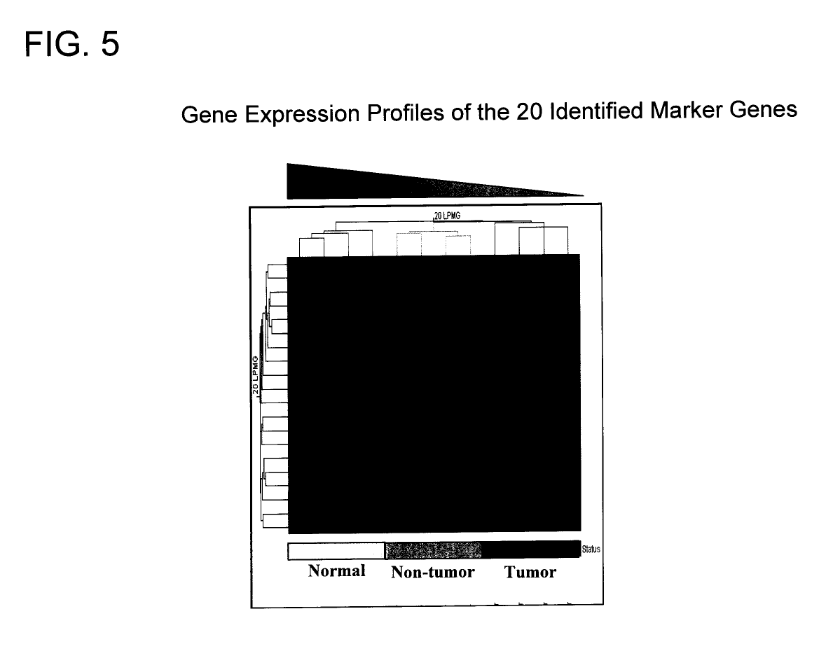

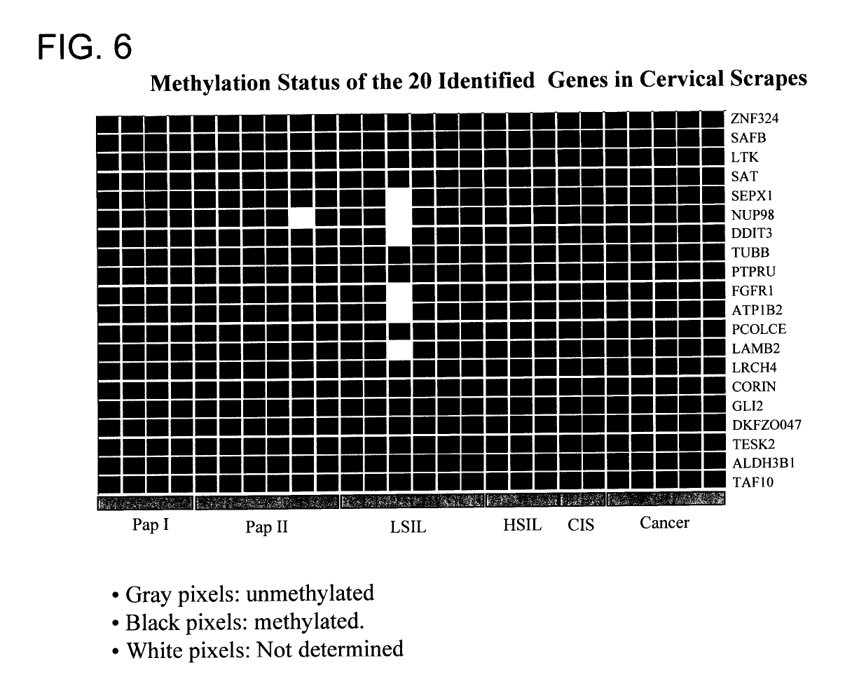

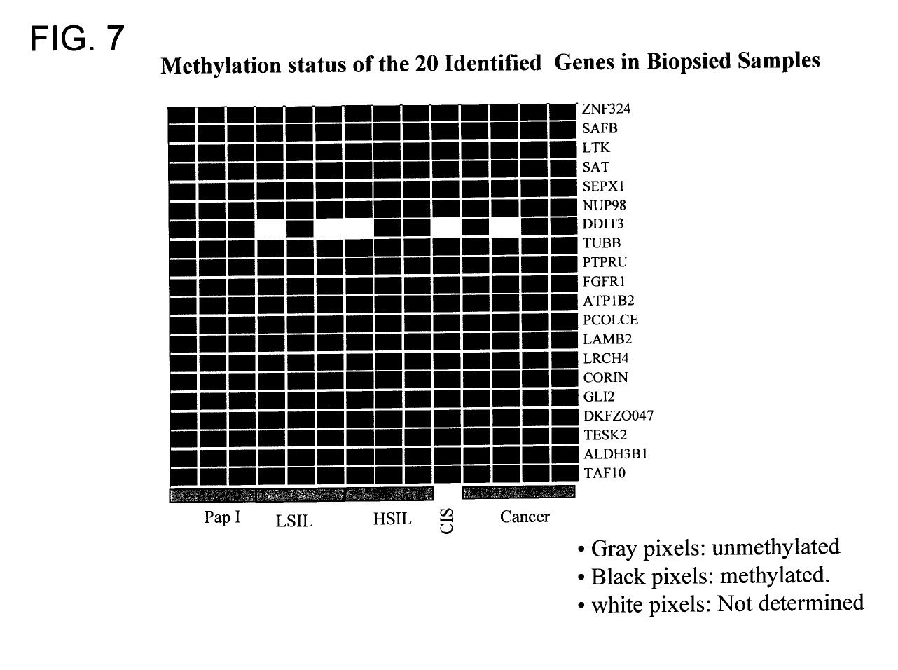

Gene expression profiles of the 20 genes were created. The expression level of the 20 genes was measured in normal, non-tumor and tumor cells (FIG. 5). Methylation status of the genes was also measured using methylation sensitive enzyme/nucleic acid sequence based amplification (NASBA) analysis such as Hpa II/MspI enzyme digestion/PCR (or enzyme digestion post-PCR) method on clinical samples and the results for the 20 genes is shown in FIG. 6 for assays from cervical scrape and FIG. 7 for assays from biopsy sample.

Thus, one aspect of the invention is in part based upon the discovery of the relationship between cervical cancer and the above 20 exemplified promoter hypermethylation of the following genes: Nucleoporin 98 kDa, Selenoprotein X, 1, DKFZP4340047 protein, Zinc finger protein 324, Testis-specific kinase 2, Corin, serine protease, GLI-Kruppel family member GLI2, Spermidine/spermine N1-acetyltransferase, Scaffold attachement factor B, Leucine-rich repeats and calponin homology (CH) domain containing 4, Laminin, beta 2 (Laminin S), ATPase Na+/K+ transporting, beta 2 polypeptide, Tubulin, beta polypeptide, Aldehyde dehydrogenase 3 family, member B1, Leukocyte tyrosine kinase, Procollagen C endopeptiase enhancer, Protein tyrosine phosphatase, receptor type, U. TAF10 RNA polymerase II, TATA box binding protein (TBP)-associated factor 30 kDa, Fibroblast growth factor receptor 1 (fms-related tyrosine kinase 2, Pfeiffer syndrome), and DNA-damage-inducible transcript 3.

In another aspect, the invention provides a method of diagnosing a cellular proliferative disorder of cervical tissue in a subject comprising determining the state of methylation of one or more nucleic acids isolated from the subject, wherein the state of methylation of one or more nucleic acids as compared with the state of methylation of one or more nucleic acids from a subject not having the cellular proliferative disorder of cervical tissue is indicative of a cellular proliferative disorder of cervical tissue in the subject. A preferred nucleic acid is a CpG-containing nucleic acid, such as a CpG island.

Another embodiment of the invention provides a method of determining a predisposition to a cellular proliferative disorder of cervical tissue in a subject comprising determining the state of methylation of one or more nucleic acids isolated from the subject, wherein the nucleic acid may be Nucleoporin 98 kDa, Selenoprotein X, 1, DKFZP4340047 protein, Zinc finger protein 324, Testis-specific kinase 2, Corin, serine protease, GLI-Kruppel family member GLI2, Spermidine/spermine N1-acetyltransferase, Scaffold attachement factor B, Leucine-rich repeats and calponin homology (CH) domain containing 4, Laminin, beta 2 (Laminin S), ATPase Na+/K+ transporting, beta 2 polypeptide, Tubulin, beta polypeptide, Aldehyde dehydrogenase 3 family, member B 1, Leukocyte tyrosine kinase, Procollagen C endopeptiase enhancer, Protein tyrosine phosphatase, receptor type, U, TAF10 RNA polymerase II, TATA box binding protein (TBP)-associated factor 30 kDa, Fibroblast growth factor receptor 1 (fms-related tyrosine kinase 2, Pfeiffer syndrome), or DNA-damage-inducible transcript 3, and combinations thereof, and wherein the state of methylation of one or more nucleic acids as compared with the state of methylation of said nucleic acid from a subject not having a predisposition to the cellular proliferative disorder of cervical tissue is indicative of a cell proliferative disorder of cervical tissue in the subject.

As used herein, “predisposition” refers to an increased likelihood that an individual will have a disorder. Although a subject with a predisposition does not yet have the disorder, there exists an increased propensity to the disease.

Another embodiment of the invention provides a method for diagnosing a cellular proliferative disorder of cervical tissue in a subject comprising contacting a nucleic acid-containing specimen from the subject with an agent that provides a determination of the methylation state of nucleic acids in the specimen, and identifying the methylation state of at least one region of at least one nucleic acid, wherein the methylation state of at least one region of at least one nucleic acid that is different from the methylation state of the same region of the same nucleic acid in a subject not having the cellular proliferative disorder is indicative of a cellular proliferative disorder of cervical tissue in the subject.

The inventive method includes determining the state of methylation of one or more nucleic acids isolated from the subject. The phrases “nucleic acid” or “nucleic acid sequence” as used herein refer to an oligonucleotide, nucleotide, polynucleotide, or to a fragment of any of these, to DNA or RNA of genomic or synthetic origin which may be single-stranded or double-stranded and may represent a sense or antisense strand, peptide nucleic acid (PNA), or to any DNA-like or RNA-like material, natural or synthetic in origin. As will be understood by those of skill in the art, when the nucleic acid is RNA, the deoxynucleotides A, G, C, and T are replaced by ribonucleotides A, G, C, and U, respectively.

The nucleic acid of interest can be any nucleic acid where it is desirable to detect the presence of a differentially methylated CpG island. The CpG island is a CpG rich region of a nucleic acid sequence. The nucleic acids includes, for example, a sequence encoding the following genes (GenBank Accession Numbers are shown):

1. NUP98 (NT_009237): Nucleoporin 98 kDa;
Amplicon size: 232 bp
gcc tcgggcaaac tctttggacagacagacctt ccaagagggc cctcaggctc cgcacgtggt ctgggcggga
agtgcgggca agagactag gaagccttcc cagcagcgcc ggcggaccgg ggcaaggaaa gggtcgaata gttaccatgc
cattctgggg ccgtccctct gattggcccg agctccctca gcccgagtcg cggcgctcca aggaaggggg cggagtctc (SEQ ID NO:1)
NUP98-F: 5′- gcctcgggcaaactctttgg-3′ (SEQ ID NO:2)
NUP98-R: 5′- gagactccgcccccttcctt-3′ (SEQ ID NO:3)
2. SEPX1 (NT_037887): Selenoprotein X, 1;
Amplicon size: 246 bp
tgca aaggcggttt cacctgggcg cggaaaggct gcatgacctc cggcagctcc ggcttagta ccagggactg
gactgggggg acacgaccct acggattcgc gccgtcgccc acctggccag gccccatgtg cgcccctagc caccagggcg
cgaggggcgg caggggcggg gccttgggga agcggcctag gggtggtccc tgggagtttc catgggcggg gcctgctggg
gagatgcgat tg (SEQ ID NO:4)
SEPX1-F: 5′-tgcaaaggcggtttcacctg-3′ (SEQ ID NO:5)
SEPX1-R: 5′-caatcgcatctccccagcag-3′ (SEQ ID NO:6)
3. DKFZP4340047 (NT_010799): DKFZP434O047 protein;
Amplicon size: 203 bp
tctggt tctcgctggg gttgtcgcgg ggcctgtgac accagatcgt tttctgccca cagctgatga agcgagtgta
taagggcatt cccatgaacg tccggccga ggtgtggtca gtcctcctga acattcagga aatcaaggcg aaaaacccca
gacaataccg ggtatgctca gccacagcac aacaaacagg ccaggcc (SEQ ID NO:7)
DKFZP4340047-F: 5′- tctggttctcgctggggttg-3′ (SEQ ID NO:8)
DKFZP4340047-R: 5′- ggcctggcctgtttgttgtg-3′ (SEQ ID NO:9)
4. ZNF324 (NT_011109): Zinc finger protein 324;
Amplicon size: 231 bp
cctccgt gctcactgct ggtgttgcct cttggaacct cggcagtttc tgctttcgca cctgcagggt ttagtctccg
ggactgcttg ggaggacacc tggaatgccg aaggcaggac tacacaccgg gcgaatccac cagcgccacc aagcccttga
aaacccgagg ccgccgcgcg actccatttc ccagcgcccc gcgcggcagg ggcttggacg ttgccaagga gacc (SEQ ID NO:10)
ZNF324-F: 5′- cctccgtgctcactgctggt-3′ (SEQ ID NO:11)
ZNF324-R: 5′- ggtctccttggcaacgtcca-3′ (SEQ ID NO:12)
5. TESK2 (NT_004511): Testis-specific kinase 2;
Amplicon size: 336 bp
cgcctttcc cacacactgc ttcttctgtt tttacctcga cttccttcct attggttccc ttcgtcctcc ggagttggca
cctacggctg ttgattggct tttctggacg cccattgtgc ccaccctcat accacgtggc attcctagcg ctaattgggc aattccacta
cctcgtttat caggagttga cggttgactg gctactctga gggagggctt gagggagggg cgtggccagc cgagaccccg
cccccaaccc ccctgctggg ccgggtaggc gtttcagtct ttcgcggccc ggagctcag cagagctacc agctgccctg ttggctt
(SEQ ID NO:13)
TESK2-F: 5′-cgcctttcccacacactgct-3′ (SEQ ID NO:14)
TESK2-R: 5′-aagccaacagggcagctggt-3′ (SEQ ID NO:15)
6. COR1N (NT_006238): Corin, serine protease;
Amplicon size: 223 bp
cggg agtgaaggga gggtgtggcc cgcgggtggg atctgtagag cagacaaaat atggggcccc tggcgcttaa
agttcagttt gtctctcttg agcttggaga aaatcatccg tagtgcctcc ccgggggaca cgtagaggag agaaaagcga ccaagataaa
agtggacaga agaataagcg agacttttta tccatgaaac agtctcctgc cctcgctcc (SEQ ID NO:16)
CORIN-F: 5′- cgggagtgaagggagggtgt-3′ (SEQ ID NO:17)
CORIN-R: 5′- ggagcgagggcaggagactg-3′ (SEQ ID NO:18)
7. GLI2 (NT_022135): GLI-Kruppel family member GLI2;
Ampicon size: 204 bp
caggggaag gcccagaaat gctcctgaag catcttgctg tcaccagtgc ccctgctgag gccccagagg
gcaggtgacc tgggtgaacc tcctaacgga cagggggctt ctccgggccc tgggtcccgg gtgcccgctc ccaccccctt
agcagaattt tggagagtgt ggctgtgttt ccttcgtggt tgtttggaca gcggc (SEQ ID NO:19)
GLI2-F: 5′- caggggaaggcccagaaatg-3′ (SEQ ID NO:20)
GLI2-R: 5′- gccgctgtccaaacaaccac-3′ (SEQ ID NO:21)
8. SAT (NT_011757): Spermidine/spermine N1-acetyltransferase;
Amplicon size: 236 bp
tc ccactggcca aggagaaaag agcaaggtca cttgtcgggg ggctgcagag ggaattacct tctttcattt
gcaaatgtta ctgggggaca caccggctcc cagtagggtt tccgccaagg ctccgcgaaa cgccactaga gggcgccgct
agcgaatccc acagcgcgcc ccgctgcccc cacttttgtc ctccgggttc acacgggcgc ccggaagaga gggtggtgcc tggg
(SEQ ID NO:22)
SAT-F: 5′-tcccactggccaaggagaaa-3′ (SEQ ID NO:23)
SAT-R: 5′-cccaggcaccaccctctctt-3′ (SEQ ID NO:24)
9. SAFB (NT_011255): Scaffold attachement factor B;
Amplicon size: 196 bp
Agccctg gggcaagaac aagcaccgcc ccctttcctg taaatgctct ggagtccgcc agacacccat
tctccggagg aggaaactga ggcacagaga gaaaggcacc tgcccaaggt cacagagcca aggctcgatc
gggagcccca ggaggggtcc cggggccacc ctggtagcgg agaagaccgc acgctgagc (SEQ ID NO:25)
SAFB-F: 5′- agccctggggcaagaacaag-3′ (SEQ ID NO:26)
SAFB-R: 5′- gctcagcgtgcggtcttctc-3′ (SEQ ID NO:27)
10. LRCH4 (NT_007933): Leucine-rich repeats and calponin homology (CH) domain
containing 4;
Amplicon size: 350 bp
gaggcaggtc cagattccct tcggaggctc taattggcct tcttgacagt cactcgcctg tatctggctt acgattggtc
tttgggggac gaggactagg caccccctgc ttgcggtccc tcggcccgtt ttcctgctag tggagtgccg gtgtcccctc ctcttggttg
gacctgattg gctccacctc ccgatctgag cattgtgatt gggtgcagct gcgctgggcg ggacggcctg gagcgtcggc
cgttggcgag cgctctatcc ttgttcccct cccttctctc gtcagactcc ggcgccggag ctccaccccc actgacgggt tctgattggc
cgcttctgac (SEQ ID NO:28)
LRCH4-F(new): 5′-gaggcaggtccagattcccttc-3′ (SEQ ID NO:29)
LRCH4-R(new): 5′-gtcagaagcggccaatcagaac-3′ (SEQ ID NO:30)
11. LAMB2 (NT_022517): Laminin, beta 2 (Laminin S);
Amplicon size: 208 bp
ccatgtttc ccccagcttc ccgttcccag ggccctgggt ggggcgcgcc ccatatcccc cacccacttt cctccttctc
tccccctcca cgccgccgcg cacattccaa ccccaggctc ctgcgatccc ggcaggccaa aaagtctgga gcggataaat
agccacaaga tccggagtcg ctcgccgtag ctctggtcca ccacccaga (SEQ ID NO:31)
LAMB2-F: 5′- ccatgtttcccccagcttcc-3′ (SEQ ID NO:32)
LAMB2-R: 5′- tctgggtggtggaccagagc-3′ (SEQ ID NO:33)
12. ATP1B2 (NT_010718): ATPase Na+/K+ transporting, beta 2 polypeptide;
Amplicon size: 223 bp
accgcgc ctggcctaat tttgcatttt taagtagaga cggggtttca ccatgtgggc aaggctagtc tcgaactcct
gacttcgtga tctgcccgcc tcggcctccc aaaatgctgg gattacaggc ataagccacc gtgccggct tgagataatg attcttaaca
tgtggtatgt agagcaagtg tactcccgcc ctagactgtg agccccgtga gagatg (SEQ ID NO:34)
ATP1B-F: 5′-accgcgcctggcctaattt-3′ (SEQ ID NO:35)
ATP1B-R: 5′- catctctcacggggctcaca-3′ (SEQ ID NO:36)
13. TUBB (NT_034880): Tubulin, beta polypeptide;
Amplicon size: 253 bp
tcac gatggccagc tccttccact gccgcccagg tgccctccca ggctcctgct gcccttcgcc cagtccctcc
ggtggtcagt gccagcatgt agatcggtgg cttaaacctg cactgttgca ggagtgcctg ggatctgtgc caagagagct tgatccctgg
aggcagccac agagtgggtc cccctgtcct tggcctgcca cgacccatca atcatccgg atgcaatggc attcagccct gcctggggt
(SEQ ID NO:37)
TUBB-F: 5′-tcacgatggccagctccttc-3′ (SEQ ID NO:38)
TUBB-R: 5′-accccaggcagggctgaat-3′ (SEQ ID NO:39)
14. ALDH3B1 (NT_033903): Aldehyde dehydrogenase 3 family, member B1;
Amplicon size: 183 bp
atc gagcaagctc ggggaacgtg accgggggct gcatgcgtca gctaacagaa cagaaagttt tgcagtgctt
tctcatacaa tgtctggaat ttacagataa cacaagtagt ttaggtcagg ggttgatatt attatcactt ttttttaact accagggcca
ggtggtggcg ccaaggtctt (SEQ ID NO:40)
ALDH3B1-F: 5′- atcgagcaagctcggggaac-3′ (SEQ ID NO:41)
ALDH3B1-R: 5′- aagaccttggcgccaccac-3′ (SEQ ID NO:42)
15. LTK (NT_010194): Leukocyte tyrosine kinase;
Amplicon size: 237 bp
gccgtggc aaaatgagct gtcaacttta ggttgacagg ggtgtggccg cgaccgcaag ggcttttgtt gccgggtgga
cccaacaggg atgggctgct ggggacagct gctggtgtgg ttcggagccg cgggtaagtg ctatctggcg gtcaggggac
cgtccagcct gaggcacttt cctaggcttg ggggtggccg gtcaacaggc caggtgtcag tggttcctcc agaggcctcg a
(SEQ ID NO:43)
LTK-F: 5′- gccgtggcaaaatgagctgt-3′ (SEQ ID NO:44)
LTK-R: 5′- tcgaggcctctggaggaacc-3′ (SEQ ID NO:45)
16. PCOLCE (NT_007933): Procollagen C endopeptiase enhancer;
Amplicon size: 208 bp
tggggttact gggacggtga gtaactgccg gcccccgtcc gcaatcgggc tccctccgtc gggcgcgagg
gggcacccca gggctggggg gactaggttc ctccaacccc ggggggcccc tacacccagc cctgggctcc cgattgcggt
cccatcaccc cctcctggcg gcagaagtct cccttgcaat gtttcaggcg gggacctc (SEQ ID NO:46)
PCOLCE-F: 5′-tggggttactgggacggtga-3′ (SEQ ID NO:47)
PCOLCE-R: 5′-gaggtccccgcctgaaacat-3′ (SEQ ID NO:48)
17. PTPRU (NT_004538): Protein tyrosine phosphatase, receptor type, U;
Amplicon size: 308 bp
g cgagggctcg ttctgggtag aggcccaaat acatttaacc tgtgtagcca gaaggtagag taggaccagt
gagggctagt gagggaggta gacttgcacg tcatgaaatg aaaaactttc taaaagccgg cgtttccaaa gccccaacaa ccgctcttgg
ccaggtgatg agcgccctgt tcctggaggt atgtagaaca cgcatttgaa ggcgccaagg tacaagggat tcaggcatcg
gatgggacag aggccgggtg tccctgggtc accgtccctg agagcgctgt acgggagcta ggcgtgg (SEQ ID NO:49)
PTPRU-F: 5′-gcgagggctcgttctgggta-3′ (SEQ ID NO:50)
PTPRU-R: 5′-ccacgcctagctcccgtaca-3′ (SEQ ID NO:51)
18. TAF10 (NT_009237): TAF10 RNA polymerase II, TATA box binding protein
(TBP)-associated factor 30 kDa;
Amplicon size: 318 bp
cgtcg aagccaggtc ttgagcgtca gacagaatga ggtgtcccag agcggcggag gagagccaga cgcacgctgg
ttccggtctg gacggaatcg ccgcggagca cggcagaggc tagggcggaa tggctacggc agcgcagttc gccaaggctc
ggtctccgcc ctgcagccta tttcctctaa cccgcagatc cactatggga ggaggcgggg agcgggctgg agagcactaa
cagagacagg cggggcgagt cgtgggcgcg tgacgtcacc ccggggtgtg cgcggcgcga gcggaagcgg aagcggctct gtt
(SEQ ID NO:52)
TAF10-F: 5′-cgtcgaagccaggtcttgagc-3′ (SEQ ID NO:53)
TAF10-R: 5′-aacagagccgcttccgcttc-3′ (SEQ ID NO:54)
19. FGFR1 (NT_007995): Fibroblast growth factor receptor 1 (fms-related tyrosine
kinase 2, Pfeiffer syndrome);
Amplicon size: 180 bp
ctccaaa gcccacgcta ccaggtacaa cctcaaggct gcggcgtctc ttcacctgcc ccctagcccc caaaccgctg
ctatgtctag ggcctgacat tccggcgccc tctgggacgt gctcagatgc aggggcgcaa acgccaaagg agaccaggct
gtaggaagag aagggcagag cgc (SEQ ID NO:55)
FGFR1-F: 5′- ctccaaagcccacgctacca-3′ (SEQ ID NO:56)
FGFR1-R: 5′- gcgctctgcccttctcttcc-3′ (SEQ ID NO:57)
20. DDIT3 (NT_029419): DNA-damage-inducible transcript 3
Amplicon size: 155 bp
acgtcgac cccctagcga gagggagcga cgggggcggt gccgcggggc tcctgagtgg cggatgcgag
ggacggggcg gggccaatgc cggcgtgcca ctttctgatt ggtaggtttt ggggtcccgc ccctgagagg agggcaaggc catggta
(SEQ ID NO:58)
DDIT3-F: 5′-acgtcgaccccctagcgaga-3′ (SEQ ID NO:59)
DDIT3-R: 5′-taccatggccttgccctcct-3′ (SEQ ID NO:60)

The bolded “ccgg” refers to sites of methylation, which are also recognized by a methylation sensitive restriction enzyme HpaII.

Cervical Cancer Biomarker—Experiment II: Using Dysplatic Cervical Tissue and Tumor Cells to Determine Discriminating Marker for High Grade Lesion

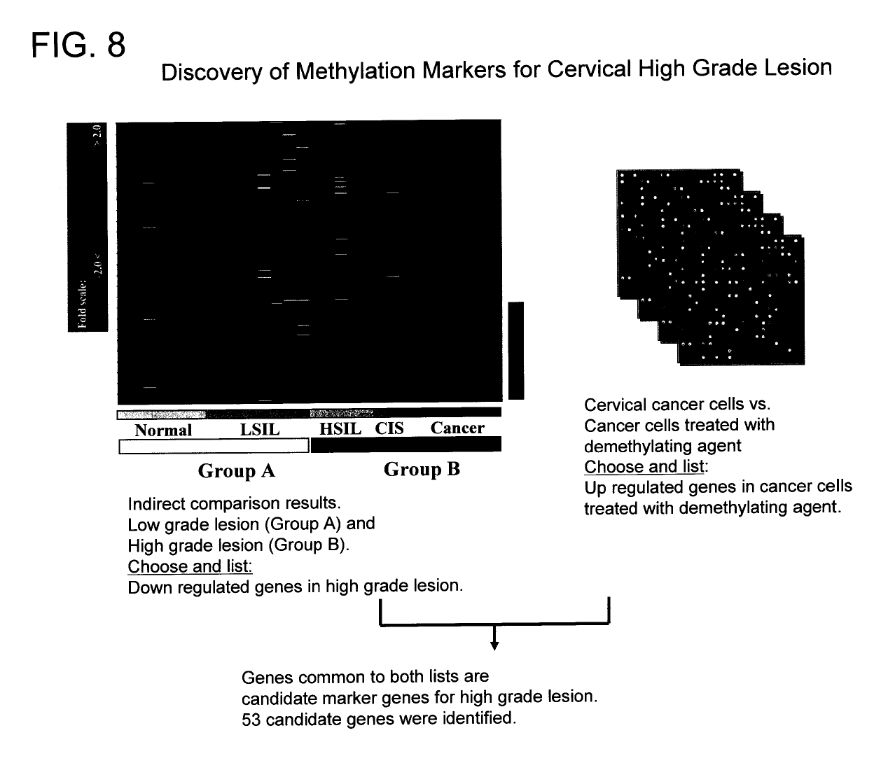

The goal of this approach is to discover methylation biomarker capable of discriminating cervical lesions, especially in low grade lesion (group A) and high grade lesion (group B) (FIG. 8).

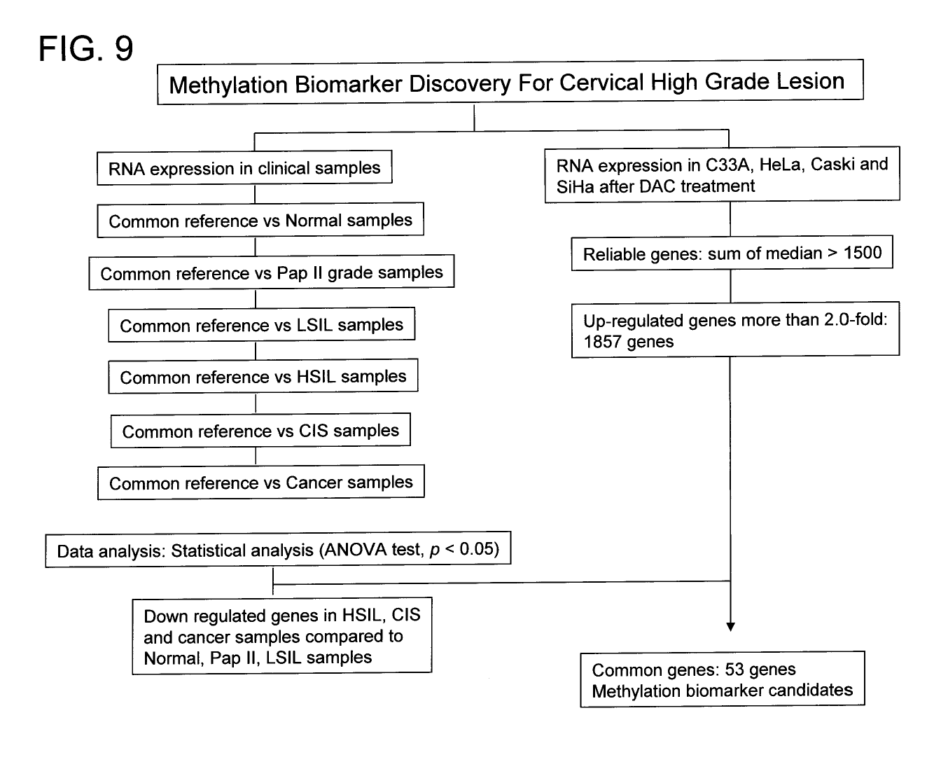

Common reference RNA was compared with normal clinical sample; common reference was compared with Pap II grade samples; common reference vs. LSIL samples; non-tumor cells vs. HSIL samples; non-tumor cells vs. CIS samples; and non-tumor cells vs. cancer samples (FIG. 9). The hybridization data were analyzed and down regulated genes in HSIL, CIS and cancer samples compared with normal, Pap II, LSIL samples were noted. Separately, cervical cancer cell lines C33A, HeLa, Caski and SiHa were treated with a demethylating agent DAC and assayed for reactivation of genes that are normally repressed in tumor cells. Overlapping genes between the noted suppressed genes in the high grade lesion gene set and the demethylation reactivated gene set were considered to be candidate genes for cervical cancer biomarkers. Fifty three (53) such overlapping genes were found. These genes were then analyzed in silico to determine whether they contained the requisite CpG island motif. A few genes (14 genes) did not contain them and were removed. Further biochemical testing was needed to determine whether the candidate genes were actually methylated when isolated from tumor cells. Methylation sensitive enzyme/nucleic acid sequence based amplification (NASBA) analysis such as Hpa II/MspI enzyme digestion/PCR (or enzyme digestion post-PCR) further removed a few other genes (11 genes) that were not methylated in any of the four cervical cancer cell lines (C33A, SiHa, HeLa and Caski). To further confirm biochemically that the candidate genes were indeed methylated in tumor cells, bisulfite sequencing assays were conducted and methylation of the final 28 genes was verified.

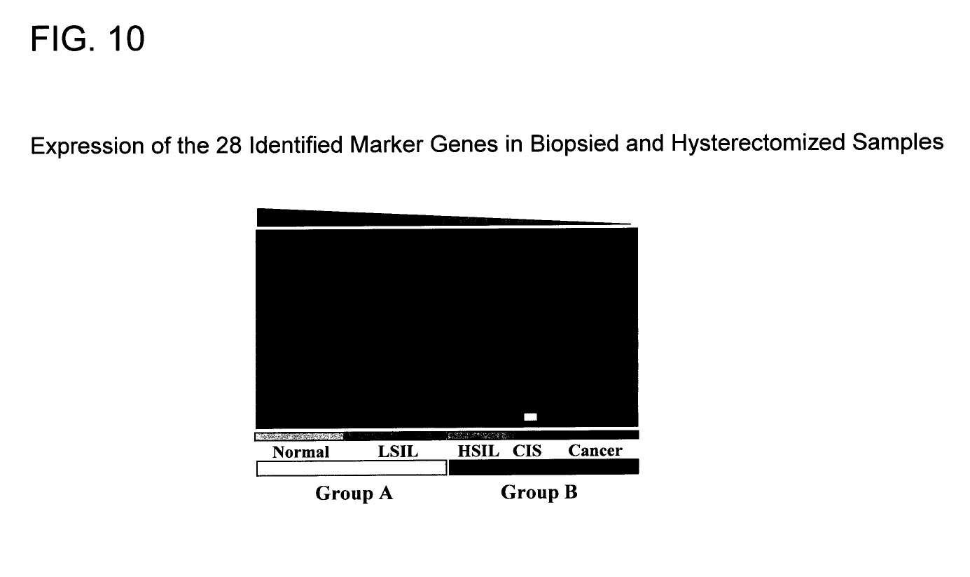

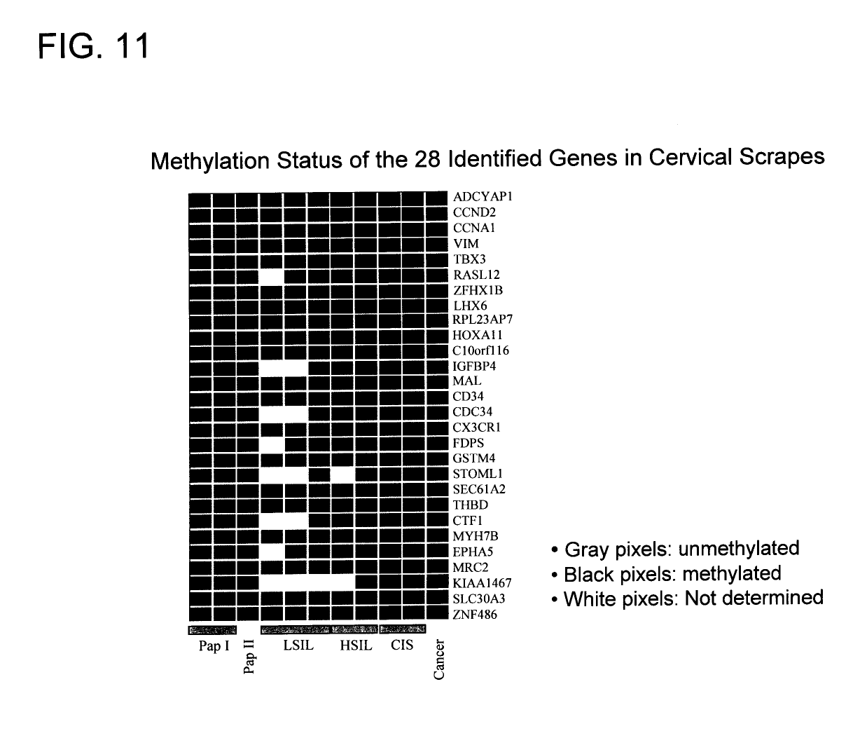

Gene expression profiles of the 28 genes were created using cervix biopsy samples. The expression level of the 28 genes was measured in normal and LSIL (Group A) and HSIL, CIS and cancer cells (Group B) (FIG. 10). Methylation status of the genes was also measured using methylation sensitive enzyme/nucleic acid sequence based amplification (NASBA) analysis such as Hpa II/MspI enzyme digestion/PCR (or enzyme digestion post-PCR) on clinical samples and the results for the 28 genes is shown in FIG. 11 for assays from cervical scrape. The methylation status assay shows that a certain demarcation can be observed for discriminating between LSIL and HSIL with these seven genes by using cervical scrapes.

Thus, one aspect of the invention is in part based upon the discovery of the ability of the biomarkers to discriminate between low grade and high grade lesions of cervical tissue dysplasia by identifying methylation markers for these grades. The exemplified marker genes include: ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin Al; CCND2 (NT_{—}009759): Cyclin D2; EPHA5 (NT_{—}022778): EphA5; HOXA11 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT_{—}086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin, and combinations thereof.

Another embodiment of the invention provides a method of determining a predisposition to a cellular proliferative disorder of cervical tissue in a subject comprising determining the state of methylation of one or more nucleic acids isolated from the subject, wherein the nucleic acid may be ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin A1; CCND2 (NT_{—}009759): Cyclin D2; EPHA5 (NT_{—}022778): EphA5; HOXA 1 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT_{—}086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin, and combinations thereof, and wherein the state of methylation of one or more nucleic acids as compared with the state of methylation of said nucleic acid from a subject not having a predisposition to the cellular proliferative disorder of cervical tissue is indicative of a cell proliferative disorder of cervical tissue in the subject.

The nucleic acid of interest can be any nucleic acid where it is desirable to detect the presence of a differentially methylated CpG island. The CpG island is a CpG rich region of a nucleic acid sequence. The nucleic acids includes, for example, a sequence encoding the following genes (GenBank Accession Numbers are shown):

1. ADCYAP1 (NT_010859): Adenylate cyclase activating polypeptide 1 (pituitary);
Amplicon size; 226 bp
caggcaggca gatgttgaca aagagggctc tccaaaaacc atgttcggat agatttttgc gaactgcaca gataaatagg
agcagaaggc cggtcacctc tgtaaccagc ggtagcagca gcagaagccg cagcttcaga ggcagccgga gagacctcgg
agcagagaag gcgccgccga ccctcgcggc tgcctggccc gcggctccta caaaggcggg ctagcc (SEQ ID NO:61)
ADCYAP 1-F; 5′- caggcaggcagatgttgacaaa-3′ (SEQ ID NO:62)
ADCYAP1-R; 5′- ggctagcccgcctttgtaggag-3′ (SEQ ID NO:63)
2. C10orf116 (NT_030059): Chromosome 10 open reading frame 116;
Amplicon size; 309 bp
ttctcctgcc tccagctcct ctctggaccc ctgtcctggc acctcttcgg tccctggttc ggtctgcccc tttcccaccg
cggcccgtct taggccagga tgtgctccct gccctgcgga ctctggagca gggcccggcc actccccgga gcctgtatga
cgggaaccgc cccgcgccct ctcccctacg cggggcaggc cagccctggg gcgccttaaa aaccggagct ggcgcttggc
atcgccactc tgggcaggat ccaacgtcgc tccagctgct cttgacgact ccacagatac cccgaagcc (SEQ ID NO:64)
C10orf116-F; 5′- ttctcctgcctccagctcctct-3′ (SEQ ID NO:65)
C10orf116-R; 5′- ggcttcggggtatctgtggagt-3′ (SEQ ID NO:66)
3. CCNA1 (NT_024524): Cyclin A1;
Amplicon size; 250 bp
ctttggggtc caggcaggtt ttggggcctc ctgtctggtg ggaggaggcc gcagcgcagc accctgctcg
tcacttggga tggagaccgg ctttcccgca atcatgtacc ctggatcttt tattgggggc tggggagaag agtatctcag ctgggaagga
ccggggctcc cagatttcgt cttccaggta acgtgggttt agtatcccga cttggaggct tgtcagaatg tttctctcct tccagcccaa
(SEQ ID NO:67)
CCNA1-F; 5′- ctttggggtccaggcaggtttt-3′ (SEQ ID NO:68)
CCNA1-R; 5′- ttgggctggaaggagagaaaca-3′ (SEQ ID NO:69)
4. CCND2 (NT_009759): Cyclin D2;
Amplicon size; 200 bp
gggccagctg ctgttctcct taataacgag aggggaaaag gagggaggga gggagagatt gaaaggagga
ggggaggacc gggaggggag gaaaggggag gaggaaccag agcggggagc gcggggagag ggaggagagc taactgccca
gccagcttgc gtcaccgctt cagagcggag aagagcgagc aggggagagc (SEQ ID NO:70)
CCND2-F; 5′- gggccagctgctgttctcctta-3′ (SEQ ID NO:71)
CCND2-R; 5′- gctctcccctgctcgctcttct-3′ (SEQ ID NO:72)
5. EPHA5 (NT_022778): EphA5;
Amplicon size; 203 bp
accctctcgacacccttgatccgagtccagatctgcactagcaaccagaactaatatttcatttaaccccaccaaagggggaggc
gagaggagccagaagcaaacttcagctgtctcagccggatccgtggttcctacatttggaggagccgcgtgccagaaggcgtaggaccccaag
gggggacaaggaggactcccgagtc (SEQ ID NO:73)
EPHA5-F; 5′- accctctcgacacccttgatcc-3′ (SEQ ID NO:74)
EPHA5-R; 5′- gactcgggagtcctccttgtcc-3′ (SEQ ID NO:75)
6. HOXA11 (NT_007819): Homeo box A11
Amplicon size; 201 bp
gaggtggggacgagagttgagctctcaccgccctctgcacactcgagaacgaggaccctgcaattgagcacaagcatgctgc
atgggggcgcaccccagcctctccgcgcgcgccgggaggccccccagccaacatgagttacaccggcgattacgtgctttcggtgagaacac
cgagtgacgatctgttgcttcccctga (SEQ ID NO:76)
HOXA11-F; 5′- gaggtggggacgagagttgagc-3′ (SEQ ID NO:77)
HOXA11-R; 5′- tcaggggaagcaacagatcgtc-3′ (SEQ ID NO:78)
7. IGFBP4 (NT_010755): Insulin-like growth factor binding protein 4;
Amplicon size; 213 bp
aagtccctttctcggtgggagactgaggccgccttggcggggcgggacgagactcctccgaggtcgggaaagggggccccg
cagcagccccttggcttcccttctcccttgcctcccctccggggctccggttcagaggcactctgggcgcctgctacagcttccaaactgcgccgc
ttccttcttcggcagaaaaggactttcagatgcggc (SEQ ID NO:79)
IGFBP4-F; 5′- aagtccctttctcggtgggaga-3′ (SEQ ID NO:80)
IGFBP4-R; 5′- gccgcatctgaaagtccttttct-3′ (SEQ ID NO:81)
8. KIAA1467 (NT_009714): KIAA1467
Amplicon size: 245 bp
cca aggccacgtc tctacgcttc cgaacagcga gttcttgatc atgcccaaca tgttgtagcc gagacgctcc
cgacgcacgg gaggacgtga ggtggcgggg gcgacggagc accacgggca gcgaccaccg gcggcagggc ggcagggcgg
cagggcggca gggcggcagg gtggcagggc ggcaaggcgg cgggacggcg aggcggcgag gcgagaggcg gggctagagg
caggggccag ag (SEQ ID NO:82)
KIAA1467-F: 5′-ccaaggccacgtctctacgc-3′ (SEQ ID NO:83)
KIAA1467-R: 5′- ctctggcccctgcctctagc-3′ (SEQ ID NO:84)
9. LHX6 (NT_008470): LIM homeobox 6;
Amplicon size; 219 bp
gtgctttttcctccccttgagcgcctctcttttctctttttggtcccgtttcgccccgatctcgctctctttttgctccgggtttccctccga
ctggccctcgaaaggcgcctgaatccgtgtcaatatagctgcttcaatttcgccgcgcgtgtcaggcgggcgggcgggcgggtgctcaccgcg
ctcggggttttcttttcttcaaccaccctccgc (SEQ ID NO:85)
LHX6-F; 5′- gtgctttttcctccccttgagc-3′ (SEQ ID NO:86)
LHX6-R; 5′- gcggagggtggttgaagaaaag-3′ (SEQ ID NO:87)
10. MAL (NT_026970): Mal, T-cell differentiation protein
Amplicon size; 204 bp
ctgtggcggtggtccagttccgccaggaaaccgccgcctggagctgtgggtcgcgcacattaacgcatccagcggaaaaatg
aaggagacccaaattcaaagttaaagtaatggtgacccgagaggtgccttgatgagaaggtttggggtccggttactgatggttatcattcttacg
agatgctggtcacctacgaagggag (SEQ ID NO:88)
MAL-F; 5′- ctgtggcggtggtccagttc-3′ (SEQ ID NO:89)
MAL-R; 5′- ctcccttcgtaggtgaccagca-3′ (SEQ ID NO:90)
11. MRC2 (NT_010783): Mannose receptor, C type 2;
Amplicon size; 200 bp
cactgacacaggggtcacgaaaatgctaaaacaagacgtgcaagaattgttgcgctccgcacacgtaacaagggctctgcctc
cccctcccccaatcttcgccctggtggccaggccctgggctccgccccttccccaccagaccgggggaggggtcctcctcggcatggaggtag
gggatgctaggctgctggagacgc (SEQ ID NO:91)
MRC2-F; 5′- cactgacacaggggtcacgaaa-3′ (SEQ ID NO:92)
MRC2-R; 5′- gcgtctccagcagcctagcat-3′ (SEQ ID NO:93)
12. RASL12 (NT_010194): RAS-like, family 12;
Amplicon size; 234 bp
aaagggcaaggcttggaattgaacccaggaagcttcctggaggaggaggcggggctggcttggtagaacaaagggaggggt
ggattttcttgctggtgaaaagagattgagtgggaggttcagggaagagtgcaaacatccttcccctctctccccctagccctggggctccttctct
gctccttcccggccgggtttgggggcgctcgggagggtgacggcagggtcctcgag (SEQ ID NO:94)
RASL12-F; 5′- aaagggcaaggcttggaattga-3′ (SEQ ID NO:95)
RASL12-R; 5′- ctcgaggaccctgccgtcac-3′ (SEQ ID NO:96)
13. RPL23AP7 (MGC70863, NT_011526): SIMILAR TO RIBOSOMAL PROTEIN
L23A
Amplicon size: 200 bp
ctcc gagccacatg caggatataa tctcccggtg tgccgttttt taagcccgtt ggaaaagcgc agtattaggg
tgggagtgac ccgattttcc aggtgccgtc tgtcgcccct ttctttgact cggaaaggga actccctgac cccttgcgct tcccgagtga
ggcaatgcct cgccctgctt cagctcgtgc acggtg (SEQ ID NO:97)
RPL23AP7-F: 5′-ctccgagccacatgcaggat-3′ (SEQ ID NO:98)
RPL23AP7-R: 5′-caccgtgcacgagctgaa-3′ (SEQ ID NO:99)
14. SLC30A3 (NT_022184): Solute carrier family 30 (zinc transporter), member 3
Amplicon size: 184 bp
tgggtc cgctcctgtt tctgcctccg cgtgagggtt gcggccacag ggtgacggcg tggtgggcga cacccccgcg
caggcatgca cagagtggtg gtcgttgggc gaaggttggt cgtgaggttc ttgggcttca ggagcgaggc ttggaaccgg
tgggcgggac tgtgtgcaga ggtggggc (SEQ ID NO:100)
SLC30A3-F: 5′-tgggtccgctcctgtttctg -3′ (SEQ ID NO:101)
SLC30A3-R: 5′-gccccacctctgcacacagt-3′ (SEQ ID NO:102)
15. TBX3 (NT_009775): T-box 3 (ulnar mammary syndrome)
Amplicon size: 177 bp
cagtgtgttg gcgcgtgttc gagtacagat acaccggggg tgtttgggta cccgcacatg gctgcgggtg
gggcgcagtg gagaggaagc acacatgc gtgtgctgag atatggccgc atccttgtgc tcccccagcc cagacgcagg
ggagaccagc accgagacac ccgagct (SEQ ID NO:103)
TBX3-F: 5′-cagtgtgttggcgcgtgttc-3′ (SEQ ID NO:104)
TBX3-R: 5′-agctcgggtgtctcggtgct-3′ (SEQ ID NO:105)
16. VIM (NT_077569): Vimentin
Amplicon size: 196 bp
cca gcccagcgct gaagtaacgg gaccatgccc agtcccaggc cccggagcag gaaggctcga gggcgccccc
accccacccg cccaccctcc ccgcttctcg ctaggtccct attggctggc gcgctccgcg gctgggatgg cagtgggagg
ggaccctctt tcctaacggg gttataaaaa cagcgccctc ggc (SEQ ID NO:106)
VIM-F: 5′-ccagcccagcgctgaagtaa-3′ (SEQ ID NO:107)
VIM-R: 5′-gccgagggcgctgtttttat-3′ (SEQ ID NO:108)
17. ZFHX1B (NT_005058): ZFHX1B
Amplicon size: 215 bp
cctgcctccc gacactcttg gcgaggtttt tgtacagttt gctccgggag ctgtttcttc gcttccacct ttttctcccc
cacacttcgc ggcttcttca tgctttttct tctcaccatt tctggccaaa actacaaaca agacttcgca ggtaggtttt ttttcctccc
cttttctctc tttttatccc tttttggtgt gctcgtcctc catcc (SEQ ID NO:109)
ZFHX1B-F: 5′-cctgcctcccgacactcttg-3′ (SEQ ID NO:110)
ZFHX1B-R: 5′-ggatggaggacgagcacacc-3′ (SEQ ID NO:111)
18. ZNF486 (NT_011295): ZNF486
Amplicon size: 249 bp
cac cctctgtggc cctgtgtcct gtaggtattg ggagatccac agccaagatg ccgggacccc ttagaagcct
agaaatggtg agagtgccca ttggacatcc tgagagaggg gagggactgg ttgatgggaa gtggctgtgg agggactcag
gcctccccgt agtcagctcc acaatctgcg tccggacttc tccttaccca gctcggcctc agtccccttc agccataaga tggtggctgc
gctgac (SEQ ID NO:112)
ZNF486-F: 5′-caccctctgtggccctgtgt-3′ (SEQ ID NO:113)
ZNF486-R: 5′-gtcagcgcagccaccatctt-3′ (SEQ ID NO:114)
19. CD34 (NT_021877): CD34 antigen
Amplicon size; 245 bp
tgagtttgctgcgtgagtaccgcccgcgcgccgcggccgcttggcttcgccgcggggagggtggaggctttctgggaggctg
aacagcagagcagagtctcacggagggaagggacccctgcccaacccacgcactgccgcccacagctgcttccccccggggccagcgcctc
acctgggagctgacgggggtgggaggggaagggaaggccatcacccccgcgagtgtgcgttagccgaggtgt (SEQ ID NO:115)
CD34-F; 5′- tgagtttgctgcgtgagtaccg-3′ (SEQ ID NO:116)
CD34-R; 5′- acacctcggctaacgcacactc-3′ (SEQ ID NO:117)
20. CDC34 (NT_011255): Cell division cycle 34
Amplicon size; 282 bp
acaaaggtcaggctgggaggagggcacagccaggctcgggctcagcttggggtgggggctccgtggggctggcgcctctcc
cgggtcaggtgctgagccgatgctggccggcgtaggcctctttcccagtttctctggggggcaggagcaccttgccaacctctgatggctccagc
gcctgcactcgcctcacaccacatggtgacttcaacggggaccagggaagccctcgcccctcccacccctcagagccctccccgacctcgccc
acttctcagagct (SEQ ID NO:118)
CDC34-F; 5′- acaaaggtcaggctgggaggag-3′ (SEQ ID NO:119)
CDC34-R; 5′- agctctgagaagtgggcgaggt-3′ (SEQ ID NO:120)
21. CTF1 (NT_086679): Cardiotrophin 1
Amplicon size; 161 bp
gtgagaaacagcaggcgctagggtttagaggaggcctggggctgaggtttcagggacctgggctcagggcttagatcaccgg
ttcgagtacacccagggggaggactggggtcggggctggggcaggacccctgcgtccactgagtctcgggaaagaatca (SEQ ID NO:121)
CTF1-F; 5′- gtgagaaacagcaggcgctagg-3′ (SEQ ID NO:122)
CTF1-R; 5′- tgattctttcccgagactcagtgg-3′ (SEQ ID NO:123)
22. CX3CR1 (NT_022517): Chemokine (C-X3-C motif) receptor 1
Amplicon size; 231 bp
cgccttcttcttcatcggcttttttggaagcatattcttcatcaccgtcatcagcattgataggtacctggccatcgtcctggccgcca
actccatgaacaaccggaccgtgcagcatggcgtcaccatcagcctaggcgtctgggcagcagccattttggtggcagcaccccagttcatgttc
acaaagcagaaagaaaatgaatgccttggtgactaccccgaggtcct (SEQ ID NO:124)
CX3CR1-F; 5′- cgccttcttcttcatcggcttt-3′ (SEQ ID NO:125)
CX3CR1-R; 5′- aggacctcggggtagtcaccaa-3′ (SEQ ID NO:126)
23. FDPS (NT_004487): FARNESYL PYROPHOSPHATE SYNTHETASE (FPP S)
Amplicon size: 176 bp
gcc aatcagctgc ccaggaagat aatgaaaact tgagtaagac aggcagccaa agccacagcg gggacgttcc
cagcctggct cgcttccggc actgacgcct ccagccgcga cctctagact tcagcccttc cattggtcgt ccgtcacagt gccacagtgc
gccatgctca cac (SEQ ID NO:127)
FDPS-F: 5′-gccaatcagctgcccaggaa-3′ (SEQ ID NO:128)
FDPS-R: 5′-gtgtgagcatggcgcactgt-3′ (SEQ ID NO:129)
24. GSTM4 (NT_019273): Glutathione S-transferase M4
Amplicon size; 234 bp
gctccccaactcagcagagagagcacaccatcagacttctaagacttagtagccaagaagtgttgaattaaactctctgagacct
ctctttagtctgaccctggcagcctcagtctcccagagcctgtgggaactcggcagccgagaggcagaaggctgggcgacgtccggagaaga
agaaacgggggaagaacttttctcttacgatctggcttt actctcacgcgcacagcc (SEQ ID NO:130)
GSTM4-F; 5′- gctccccaactcagcagagaga-3′ (SEQ ID NO:131)
GSTM4-R; 5′- ggctgtgcgcgtgagagtaaag-3′ (SEQ ID NO:132)
25. MYH7B (NT_028392): MYOSIN, HEAVY POLYPEPTIDE 7B, CARDIAC MU
Amplicon size: 265 bp
atcgccaagt ttggcactgt aatttttttt tgagacggag tctcgtactg tcgcccaggc tggagtgcag tggtgccatc
tccactcact gcaagctctg cctcccgggt tcacgccatt ctcctgcctc agcctccaga gtagctggga ctacaggcgc ccgccaccac
ctctggctaa tttttttgta tttttactag agacggggtt tcactgtgtt agccaggatg gtctcgatct cctgacctcg tgatccgccc
tcctc (SEQ ID NO:133)
MYH7B-F: 5′-atcgccaagtttggcactgt-3′ (SEQ ID NO:134)
MYH7B-R: 5′-gaggagggcggatcacga-3′ (SEQ ID NO:135)
26. SEC61A2 (NT_077569): Sec61 alpha 2 subunit (S. cerevisiae)
Amplicon size; 340 bp
tttggagaagacggaggggactagcttacgtcgagacagaggagttcataactattaactatacacaacgacgtgtcacaaatgt
ttacaaacttccaagagataacgataagaagcggccgggcgcggtggctcacgcctgtaatcccagcactttgggaggccgaggcgggcgga
tcacgaggtcaggagatcgagaccatcctggctaacacggtgaaaccccgtctctactaaaaatacaaaaaattagccgggcgtggtggcggg
cgcctgtagccccagctactcgggaggctgaggcaggagaatggcgtgaacctacctgggaggcggagtt (SEQ ID NO:136)
SEC61A2-F; 5′- tttggagaagacggaggggact-3′ (SEQ ID NO:137)
SEC61A2-R; 5′- aactccgcctcccaggtaggtt-3′ (SEQ ID NO:138)
27. STOML1 (NT_010194): Stomatin (EPB72)-like 1
Amplicon size: 244 bp
cgccgatcac acccagtagc cctgggctgc agttcggtcc tcccagcgca gggggagcca tgcgacggcg
gaagcgggcc ccggccggcc tcctcttcct gcgcccgcgc cgccgcgggt ggccgcgcgg gtgaagccgg aagcagcagc
caggagggcg gggccgcgta gggcccactg gccagggagg gcgccgcgcg gaggcgcggg gcgtgtctcc tgtcaaaagc
catgctcggc aggt (SEQ ID NO:139)
STOML1-F: 5′-cgccgatcacacccagtagc-3′ (SEQ ID NO:140)
STOML1-R: 5′-acctgccgagcatggctttt-3′ (SEQ ID NO:141)
28. THBD (NT_011387): Thrombomodulin
Amplicon size: 194 bp
ccccc actccccatt caaagccctc ttctctgaag tctccggttc ccagagctct tgcaatccag gctttccttg
gaagtggctg taacatgtat gaaaagaaag aaaggaggac caagagatga aagagggctg cacgcgtggg ggcccgagtg
gtgggcgggg acagtcgtct tgttacaggg gtgctggcc (SEQ ID NO:142)
THBD-F: 5′-cccccactccccattcaaag-3′ (SEQ ID NO:143)
THBD-R: 5′-ggccagcacccctgtaacaa-3′ (SEQ ID NO:144)

Methylation

Any nucleic acid sample, in purified or nonpurified form, can be utilized in accordance with the present invention, provided it contains or is suspected of containing, a nucleic acid sequence containing a target locus (e.g., CpG-containing nucleic acid). One nucleic acid region capable of being differentially methylated is a CpG island, a sequence of nucleic acid with an increased density relative to other nucleic acid regions of the dinucleotide CpG. The CpG doublet occurs in vertebrate DNA at only about 20% of the frequency that would be expected from the proportion of G*C base pairs. In certain regions, the density of CpG doublets reaches the predicted value; it is increased by ten fold relative to the rest of the genome. CpG islands have an average G*C content of about 60%, compared with the 40% average in bulk DNA. The islands take the form of stretches of DNA typically about one to two kilobases long. There are about 45,000 such islands in the human genome.

In many genes, the CpG islands begin just upstream of a promoter and extend downstream into the transcribed region. Methylation of a CpG island at a promoter usually prevents expression of the gene. The islands can also surround the 5′ region of the coding region of the gene as well as the 3′ region of the coding region. Thus, CpG islands can be found in multiple regions of a nucleic acid sequence including upstream of coding sequences in a regulatory region including a promoter region, in the coding regions (e.g., exons), downstream of coding regions in, for example, enhancer regions, and in introns.

In general, the CpG-containing nucleic acid is DNA. However, invention methods may employ, for example, samples that contain DNA, or DNA and RNA, including messenger RNA, wherein DNA or RNA may be single stranded or double stranded, or a DNA-RNA hybrid may be included in the sample. A mixture of nucleic acids may also be employed. The specific nucleic acid sequence to be detected may be a fraction of a larger molecule or can be present initially as a discrete molecule, so that the specific sequence constitutes the entire nucleic acid. It is not necessary that the sequence to be studied be present initially in a pure form; the nucleic acid may be a minor fraction of a complex mixture, such as contained in whole human DNA. The nucleic acid-containing sample used for determination of the state of methylation of nucleic acids contained in the sample or detection of methylated CpG islands may be extracted by a variety of techniques such as that described by Sambrook, et al. (Molecular Cloning: A Laboratory Manual, Cold Spring Harbor, N.Y., 1989; incorporated in its entirety herein by reference).

A nucleic acid can contain a regulatory region which is a region of DNA that encodes information that directs or controls transcription of the nucleic acid. Regulatory regions include at least one promoter. A “promoter” is a minimal sequence sufficient to direct transcription, to render promoter-dependent gene expression controllable for cell-type specific, tissue-specific, or inducible by external signals or agents. Promoters may be located in the 5′ or 3′ regions of the gene. Promoter regions, in whole or in part, of a number of nucleic acids can be examined for sites of CG-island methylation. Moreover, it is generally recognized that methylation of the target gene promoter proceeds naturally from the outer boundary inward. Therefore, early stage of cell conversion can be detected by assaying for methylation in these outer areas of the promoter region.

Nucleic acids isolated from a subject are obtained in a biological specimen from the subject. If it is desired to detect cervical cancer or stages of cervical cancer progression, the nucleic acid may be isolated from cervical tissue by scraping or taking a biopsy. These specimen may be obtained by various medical procedures known to those of skill in the art.

In one aspect of the invention, the state of methylation in nucleic acids of the sample obtained from a subject is hypermethylation compared with the same regions of the nucleic acid in a subject not having the cellular proliferative disorder of cervical tissue. Hypermethylation, as used herein, is the presence of methylated alleles in one or more nucleic acids. Nucleic acids from a subject not having a cellular proliferative disorder of cervical tissues contain no detectable methylated alleles when the same nucleic acids are examined. Further, the various biomarker genes may be methylated at various stages of dysplasia.

Individual Genes and Panel

It is understood that the present invention may be practiced using each gene separately as a diagnostic or prognostic marker or a few marker genes combined into a panel display format so that several marker genes may be detected to increase reliability and efficiency. Further, any of the genes identified in the present application may be used individually or as a set of genes in any combination with any of the other genes that are recited in the application.

Methylation Detection Methods

Detection of Differential Methylation—Methylation Sensitive Restriction Endonuclease

Detection of differential methylation can be accomplished by contacting a nucleic acid sample with a methylation sensitive restriction endonuclease that cleaves only unmethylated CpG sites under conditions and for a time to allow cleavage of unmethylated nucleic acid. In a separate reaction, the sample is further contacted with an isoschizomer of the methylation sensitive restriction endonuclease that cleaves both methylated and unmethylated CpG-sites under conditions and for a time to allow cleavage of methylated nucleic acid. Specific primers are added to the nucleic acid sample under conditions and for a time to allow nucleic acid amplification to occur by conventional methods. The presence of amplified product in the sample digested with methylation sensitive restriction endonuclease but absence of an amplified product in sample digested with an isoschizomer of the methylation sensitive restriction enzyme endonuclease that cleaves both methylated and unmethylated CpG-sites indicates that methylation has occurred at the nucleic acid region being assayed. However, lack of amplified product in the sample digested with methylation sensitive restriction endonuclease together with lack of an amplified product in the sample digested with an isoschizomer of the methylation sensitive restriction enzyme endonuclease that cleaves both methylated and unmethylated CpG-sites indicates that methylation has not occurred at the nucleic acid region being assayed.

As used herein, a “methylation sensitive restriction endonuclease” is a restriction endonuclease that includes CG as part of its recognition site and has altered activity when the C is methylated as compared to when the C is not methylated. Preferably, the methylation sensitive restriction endonuclease has inhibited activity when the C is methylated (e.g., Smal). Specific non-limiting examples of methylation sensitive restriction endonucleases include Sma I, BssHII, or HpaII, BSTUI, and NotI. Such enzymes can be used alone or in combination. Other methylation sensitive restriction endonucleases will be known to those of skill in the art and include, but are not limited to SacI, and EagI, for example. An “isoschizomer” of a methylation sensitive restriction endonuclease is a restriction endonuclease that recognizes the same recognition site as a methylation sensitive restriction endonuclease but cleaves both methylated and unmethylated CGs, such as for example, MspI. Those of skill in the art can readily determine appropriate conditions for a restriction endonuclease to cleave a nucleic acid (see Sambrook et al., Molecular Cloning: A Laboratory Manual, Cold Spring Harbor Press, 1989).

Primers of the invention are designed to be “substantially” complementary to each strand of the locus to be amplified and include the appropriate G or C nucleotides as discussed above. This means that the primers must be sufficiently complementary to hybridize with their respective strands under conditions that allow the agent for polymerization to perform. Primers of the invention are employed in the amplification process, which is an enzymatic chain reaction that produces exponentially increasing quantities of target locus relative to the number of reaction steps involved (e.g., polymerase chain reaction (PCR)). Typically, one primer is complementary to the negative (−) strand of the locus (antisense primer) and the other is complementary to the positive (+) strand (sense primer). Annealing the primers to denatured nucleic acid followed by extension with an enzyme, such as the large fragment of DNA Polymerase I (Klenow) and nucleotides, results in newly synthesized + and − strands containing the target locus sequence. Because these newly synthesized sequences are also templates, repeated cycles of denaturing, primer annealing, and extension results in exponential production of the region (i.e., the target locus sequence) defined by the primer. The product of the chain reaction is a discrete nucleic acid duplex with termini corresponding to the ends of the specific primers employed.

Preferably, the method of amplifying is by PCR, as described herein and as is commonly used by those of ordinary skill in the art. However, alternative methods of amplification have been described and can also be employed such as real time PCR or linear amplification using isothermal enzyme. Multiplex amplification reactions may also be used.

Detection of Differential Methylation—Bifulfite Sequencing Method

Another method for detecting a methylated CpG-containing nucleic acid includes contacting a nucleic acid-containing specimen with an agent that modifies unmethylated cytosine, amplifying the CpG-containing nucleic acid in the specimen by means of CpG-specific oligonucleotide primers, wherein the oligonucleotide primers distinguish between modified methylated and non-methylated nucleic acid and detecting the methylated nucleic acid. The amplification step is optional and although desirable, is not essential. The method relies on the PCR reaction itself to distinguish between modified (e.g., chemically modified) methylated and unmethylated DNA. Such methods are described in U.S. Pat. No. 5,786,146, the contents of which are incorporated herein in their entirety especially as they relate to the bisulfite sequencing method for detection of methylated nucleic acid.

Substrates

Once the target nucleic acid region is amplified, the nucleic acid can be hybridized to a known gene probe immobilized on a solid support to detect the presence of the nucleic acid sequence.

As used herein, “substrate,” when used in reference to a substance, structure, surface or material, means a composition comprising a nonbiological, synthetic, nonliving, planar, spherical or flat surface that is not heretofore known to comprise a specific binding, hybridization or catalytic recognition site or a plurality of different recognition sites or a number of different recognition sites which exceeds the number of different molecular species comprising the surface, structure or material. The substrate may include, for example and without limitation, semiconductors, synthetic (organic) metals, synthetic semiconductors, insulators and dopants; metals, alloys, elements, compounds and minerals; synthetic, cleaved, etched, lithographed, printed, machined and microfabricated slides, devices, structures and surfaces; industrial polymers, plastics, membranes; silicon, silicates, glass, metals and ceramics; wood, paper, cardboard, cotton, wool, cloth, woven and nonwoven fibers, materials and fabrics.

Several types of membranes are known to one of skill in the art for adhesion of nucleic acid sequences. Specific non-limiting examples of these membranes include nitrocellulose or other membranes used for detection of gene expression such as polyvinylchloride, diazotized paper and other commercially available membranes such as GENESCREEN™, ZETAPROBE™ (Biorad), and NYTRAN™. Beads, glass, wafer and metal substrates are included. Methods for attaching nucleic acids to these objects are well known to one of skill in the art. Alternatively, screening can be done in liquid phase.

Hybridization Conditions

In nucleic acid hybridization reactions, the conditions used to achieve a particular level of stringency will vary, depending on the nature of the nucleic acids being hybridized. For example, the length, degree of complementarity, nucleotide sequence composition (e.g., GC v. AT content), and nucleic acid type (e.g., RNA v. DNA) of the hybridizing regions of the nucleic acids can be considered in selecting hybridization conditions. An additional consideration is whether one of the nucleic acids is immobilized, for example, on a filter.

An example of progressively higher stringency conditions is as follows: 2×SSC/0.1% SDS at about room temperature (hybridization conditions); 0.2×SSC/0.1% SDS at about room temperature (low stringency conditions); 0.2×SSC/0.1% SDS at about 42.degree. C. (moderate stringency conditions); and 0.1.times.SSC at about 68° C. (high stringency conditions). Washing can be carried out using only one of these conditions, e.g., high stringency conditions, or each of the conditions can be used, e.g., for 10-15 minutes each, in the order listed above, repeating any or all of the steps listed. However, as mentioned above, optimal conditions will vary, depending on the particular hybridization reaction involved, and can be determined empirically. In general, conditions of high stringency are used for the hybridization of the probe of interest.

Label

The probe of interest can be detectably labeled, for example, with a radioisotope, a fluorescent compound, a bioluminescent compound, a chemiluminescent compound, a metal chelator, or an enzyme. Those of ordinary skill in the art will know of other suitable labels for binding to the probe, or will be able to ascertain such, using routine experimentation.

Kit

Invention methods are ideally suited for the preparation of a kit. Therefore, in accordance with another embodiment of the present invention, there is provided a kit useful for the detection of a cellular proliferative disorder in a subject. Invention kits include a carrier means compartmentalized to receive a sample therein, one or more containers comprising a first container containing a reagent which sensitively cleaves unmethylated cytosine, a second container containing primers for amplification of a CpG-containing nucleic acid, and a third container containing a means to detect the presence of cleaved or uncleaved nucleic acid. Primers contemplated for use in accordance with the invention include those set forth in SEQ ID NOS:1-144, and any functional combination and fragments thereof. Functional combination or fragment refers to its ability to be used as a primer to detect whether methylation has occurred on the region of the genome sought to be detected.

Carrier means are suited for containing one or more container means such as vials, tubes, and the like, each of the container means comprising one of the separate elements to be used in the method. In view of the description provided herein of invention methods, those of skill in the art can readily determine the apportionment of the necessary reagents among the container means. For example, one of the container means can comprise a container containing methylation sensitive restriction endonuclease. One or more container means can also be included comprising a primer complementary to the locus of interest. In addition, one or more container means can also be included containing an isoschizomer of the methylation sensitive restriction enzyme.

The present invention is not to be limited in scope by the specific embodiments described herein. Indeed, various modifications of the invention in addition to those described herein will become apparent to theose skilled in the art from the foregoing description and accompanying figures. Such modifications are intended to fall within the scope of the appended claims. The following examples are offered by way of illustration of the present invention, and not by way of limitation.

Other Biomarker Development

Gene Expression Profiling

Applicants have developed optimal conditions for collecting and maintaining cervical scraped cells without disturbing the quality of the mRNA. In addition, applicants have developed a reliable RNA linear amplification and labeling method. Using these applicants were able to generate statistically fully acceptable microarray data set with cervical scrapes composed of a small number of various cell types. The collected cell mass includes for example, cervical scrapes, exfoliated cells by brush during routine screening Pap smears, which is composed of different cell types depending on the cell cytological stage according to the Bethesda system of cytological classification.

Differential expression may be seen at the RNA level if its RNA transcript varies in abundance between different samples. An expression profile may be defined as a dataset that contains information reflecting the absolute or relative expression level of a plurality of genes in a biological sample. Thus, the biological sample may range from a single cell to a complex population of cells such as found in cervical scraped specimen. Generally, an expression profile contains measurements of the expression level of dozens, hundreds, or even thousands of genes. In general, an expression profile reflecting the absolute or relative expression level of an appropriately selected set of genes in a pure population of cells of a particular type constitutes a pure cell type signature for that cell type. Therefore, gene expression profiling using microarray technology offers the opportunity to rapidly and efficiently quantify gene expression patterns of over thousands of genes. Gene expression profiling has been applied to a large number of different cell types.

Cervical lesion classification study in the present application with promoter methylation profile provides insight into the development of gene expression profile itself as a classifier (being capable of discriminating one cytological stage from others) of diagnostic classification. It is possible to identify certain genes that are more highly expressed in a certain stage. Accordingly, gene expression profiling to distinguish between normal/benign change and precancerous lesions can be carried out.

HPV Viral RNA Detection in Combination With Other Detection Methods

HPV DNA tests such as Digene® (Gaithersburg, Md.) and HPV genotyping DNA chip can detect the presence of many HPV types but not the behavior of the virus, and whether it is normal or not. The main problems of conventional HPV DNA test include: (1) missed detection of a small and may be undetectable quantities of HPV DNA, which produces large amount of both E6/E7 mRNA and oncoproteins; and (2) conventional HPV DNA test indentifies 20-30% of normal women as positive. More than 90% of these will be false positive answers because there is no production of oncoproteins. Thus, for conventional diagnostic systems, the specificity of an HPV DNA test will not be higher than 70-80%.

In most cervical carcinoma cases HPV is integrated. It has been shown that integrated HPV DNA produce stabilized viral oncogenes, E6/E7 mRNAs. It has also been shown that persistent oncogene expression may only happen when HPV is integrated. Loss of HPV replication may also be linked to HPV integration. Increasing body of evidence shows a tight correlation between the expression of oncoproteins E6/E7 and the development of cervical cancer.

The availability of specific diagnostic markers to discriminate between patients with clinically benign atypical changes from those with cancer and high-grade dysplasia would be very useful. Recently, specific assay for the detection of viral RNAs (E6/E7) have been reported using nucleic acid sequence based amplification (NASBA) measuring 5 different types of viral oncogenes at once such as PreTect HPV-Proofer assay (NorChip, Norway) and real time quantitative RT-PCR methods. Considering the number of high risk types of HPV, it is necessary to develop a DNA chip that detects not only viral RNAs (E6/E7 mRNAs) but that which genotypes the RNA origin.

We have obtained successful results with DNA chip-based assay that shows a strong potential for these. Total RNA was extracted from cervical cell line, HeLa, integrated with HPV type 18, and labeled with an RNA linear amplification method. Labeled target was hybridized onto DNA chip containing HPV gene probes specific for type 18 E6 and E7 as well as several gene probes associated with cervical cancer cell line. Results showed a clear signal for the presence of viral RNAs.

Accordingly, combinatorial clinical cohort study that measures the correlation between the presence of viral RNAs, HPV DNA and distinctive expression pattern and methylation status, Pap smear (cytology), histology, and cervical lesion status provides a reliable score, which utilizes multiple biomarkers.

### EXAMPLES

### Example 1

### Identification of Genes Repressed in Cervical Cancer

To identify genes repressed in cervical cancer, microarray hybridization experiments were carried out. Microarray hybridizations were performed according to standard protocol (Schena et al, 1995, Science, 270: 467-470). Total RNA was isolated from three different types (normal cervical part of hysterectomized tissues (4 samples), non-tumor adjacent to tumor part (4 samples) and tumor part (4 samples) of cervical cancer patients) of tissues. To compare relative difference in gene expression level of three different types of tissues indirectly, we prepared common reference RNA (indirect comparison). Total RNA was isolated from 11 human cancer cell lines. Total RNA from cell lines and cervical tissues was isolated using Tri Reagent (Sigma, USA) according to manufacturer's instructions. To make common reference RNA, equal amounts of total RNA from 11 cancer cell lines were combined. The common reference RNA was used as an internal control. To compare relative difference in gene expression levels among three different types of tissues, RNAs isolated from normal, non-tumor and tumor tissues were indirectly compared with common reference RNA. 2 ug of total RNA was labeled with Cy3-dUTP or Cy5-dUTP using Amino Allyl MessageAmp aRNA kit (Ambion). The common referene RNA was labeled with Cy3 and RNA from cervical tissues was labeled with Cy5. Both Cy3- and Cy5-labeled cDNA were purified using PCR purification kit (Qiagen, Germany). The purified cDNA was combined and concentrated at a final volume of 27 ul using Microcon YM-30 (Millipore Corp., USA).

Total 80 ul of hybridization mixture contained: 27 ul labeled cDNA targets, 20 ul of 20×SSC, 8 ul of 1% SDS, 24 ul of formamide (Sigma, USA) and 20 ug of human Cotl DNA (Invitrogen Corp., USA). The hybridization mixtures were heated at 100° C. for 2 min and immediately hybridized to human 22K oligonucleotide (Illumina, USA) microarrays. The arrays were hybridized at 42° C. for 12-16 h in the humidified HybChamber X (GenomicTree, Inc., Korea). After hybridization, microarray slides were imaged using Axon 4000B scanner (Axon Instruments Inc., USA). The signal and background fluorescence intensities were calculated for each probe spot by averaging the intensities of every pixel inside the target region using GenePix Pro 4.0 software (Axon Instruments Inc., USA). Spots were excluded from analysis due to obvious abnormalities. All data normalization, statistical analysis and cluster analysis were performed using GeneSpring 7.2 (Agilent, USA).

To determine relative difference in gene expression levels among normal, non-tumor and tumor tissues, statistical analysis (ANOVA, p<0.01) was performed. From the results of statistical analysis, following three groups of genes were extracted: 1) 380 genes that are repressed in non-tumor part tissues compared with cervical normal specimens, 2) 286 genes that are repressed in tumor tissues compared with cervical normal specimens, and 3) 746 genes that are repressed in tumor tissues compared with non-tumor samples. Consequently, 1200 tumor repressed genes were obtained after removing redundant genes.

### Example 2

### Identification of Methylation Controlled Gene Expression

To determine whether the expression of any of the genes identified in Example 1 is controlled by promoter methylation, cervical cancer cell line C33A was treated with demethylation agent, 5-aza-2′ deoxycytidine (DAC, Sigma, USA) for 4-5 days at a concentration of 1.0 uM. Cells were harvested and total RNA was isolated from treated and untreated cell lines using Tri reagent. To determine gene expression changes by DAC treatment, transcript level between untreated and treated cell lines was directly compared. From this experiment, 417 genes have been identified that show elevated expression when treated with DAC compared with the control group which was not treated with DAC. When the list of 1200 tumor repressed genes was compared with the list of 417 demethylation reactivated genes, 29 common genes were found.

### Example 3

### Confirmation of Methylation of Identified Genes

### Example 3.1

### In Silico Analysis of CpG Island in Promoter Region

The promoter regions of the 29 genes were scanned for the presence of CpG islands using MethPrimer (http://itsa.ucsf.edu/˜urolab/methprimer/index1.html). Five genes did not contain the CpG island and were dropped from the common gene list.

### Example 3.2

### Biochemical Assay for Methylation

To biochemically determine the methylation status of the remaining 24 genes, methylation status of each promoter was detected using the characteristics of restriction endonucleases, HpaII (methylation-sensitive) and MspI (methylation-insensitive) followed by PCR. Both enzymes recognize the same DNA sequence, 5′-CCGG-3′. HpaII is inactive when internal cytosine residue is methylated, whereas MspI is active regardless of whether the internal cytosine residue is methylated or not. In the case that the cytosine residue at the CpG site is unmethylated, both enzymes can digest the target sequence. To determine the methylation status of a specific gene, PCR targets containing one or more HpaII sites from CpG islands in the promoter region were selected. 100 ng of genomic DNA from cervical cancer cell lines C33A, HeLa, Caski, and SiHa were digested with 5 U of HpaII and 10 U of MspI, respectively and purified using Qiagen PCR purification kit. Specific primers were used to amplify regions of interest. 5 ng of the purified genomic DNA was amplified by PCR using gene-specific primer sets. DNA from undigested control sample was amplified to determine PCR adequacy. The PCR was performed as follows: 94 ° C, 1 min; 66° C., 1 min; 72° C., 1 min (30 cycles); and 72° C., 10 min for final extension. Each amplicon was separated on a 2% agarose gel containing ethidium bromide. If the band density of HpaII amplicon is 1.5-fold greater than that of MspI amplicon, the target region was considered to be methylated, while less than 1.5-fold was considered to be unmethylated. From this, it was discovered that 4 genes were not methylated, leaving 20 confirmed candidate genes that fit the criteria of being down regulated in tumor, up regulated under demethylation conditions, contains a CpG island in its promoter and is actually methylated in the cancer cell lines.

### Example 3.3

### Bisulfite Sequencing of Methylated Promoter

To further confirm the methylation status of the 20 identified genes, the inventors performed bisulfite sequencing of the individual promoters. Upon treatment of the DNA with bisulfite, unmethylated cytosine is modified to uracil and the methylated cytosine undergoes no change. The inventors performed the bisulfite modification according to Sato, N. et al., Cancer Research, 63:3735, 2003, the contents of which are incorporated by reference herein in its entirety especially regarding the use of bisulfite modification method as applied to detect DNA methylation. The bisulfite treatment was performed on 1 μg of the genomic DNA of the cervical cancer cell line C33A using MSP (Methylation-Specific PCR) bisulfite modification kit (In2Gen, Inc., Seoul, Korea). After amplifying the bisulfite-treated C33A genomic DNA by PCR, the nucleotide sequence of the PCR products was analyzed. The results confirmed that the genes were all methylated.

### Example 4

### Gene Expression Profile of the Identified Genes

FIG. 5 shows the gene expression profiles of the 20 genes that were identified. As shown in FIG. 5, gene expression was repressed in the non-tumor and tumor tissues compared with the cervical normal specimens. Further, gene expression was more repressed in the tumor tissues compared with the non-tumor tissues.

### Example 5

### Promoter Methylation Assay on Clinical Samples

To determine the clinical applicability of the methylated promoters of the 20 selected genes of the present invention, methylation assay was performed with cervical scrapes and cervix biopsy clinical samples. Methylation assay was performed as described supra using the restriction enzyme/PCR method.

FIG. 6 shows the results of the methylation assay on cervical scrapes as diagnosed with Pap system and Bethesda system of cytological indicators. As shown in FIG. 6, most of the 20 gene markers were not methylated in the normal clinical samples. However, the number of methylated genes gradually increased as the normal cells progressed in severity reaching to cancer. Almost all of the genes were methylated in cancer samples but not in normal cells as predicted.

The same experiments were performed with clinical cervical biopsy and tissue samples. As shown in FIG. 7, only a few of the 20 gene promoters were methylated among the normal samples (endometriosis or myoma of uterus). However, as the cancer progresses, many genes were methylated. 19 of the 20 genes were methylated among the cancer samples. Therefore, the promoters of the 20 genes are useful for hierarchical clustering analysis and significant differentiation of normal cells to cancer cells.

### Example 6

### Identification of Genes Repressed in Higher Grade of Cervical Lesions

To determine the relative gene expression changes in various grades of lesions (normal, LSIL, HSIL, CIS and cancer) in cervical clinical samples, microarray hybridization was performed using 34K human oligonucleotide (Qiagen) microarray. Microarray experiments were performed according to standard protocol (Schena et al, 1995, Science, 270: 467-470). To determine gene expression levels in various grades of cervical lesions, we prepared total RNA from normal (7 samples), LSIL (8 samples), HSIL (5 samples), CIS (2 samples) and cancer (8 samples) tissue samples. To compare relative gene expression levels across samples, RNA isolated from clinical cervical biopsy or hysterectomized samples were indirectly compared with common reference RNA including a mixture of equal amounts of total RNA from 11 human cancer cell lines. Total RNA from cell lines and cervical tissues was isolated using Tri Reagent (Sigmal, USA) according to manufacturer's instructions. Microarray experiment was conducted with a total of 30 clinical samples including normal (7 samples), LSIL (8 samples), HSIL (5 samples), CIS (2 samples) and cancer (8 samples) tissue samples. 2 ug of RNA was labeled with Cy3-dUTP or Cy5-dUTP respectively using Amino Allyl MessageAmp aRNA kit (Ambion). The common reference RNA was labeled with Cy3 and RNAs from various grades of cervical tissues were labeled with Cy5. Both Cy3- and Cy5-labeled cDNA were purified using PCR purification kit (Qiagen, Germany). The purified cDNA was combined and concentrated at a final volume of 27 ul using Microcon YM-30 (Millipore Corp., USA).

Total 80 ul of hybridization mixture contained: 27 ul labeled cDNA targets, 20 ul of 20×SSC, 8 ul of 1% SDS, 24 ul of formamide (Sigma, USA) and 20 ug of human Cot1 DNA (Invitrogen Corp., USA). The hybridization mixtures were heated at 100° C. for 2 min and immediately hybridized to human 34K oligonucleotide microarrays. The arrays were hybridized at 42° C. for 12-16 h in the humidified HybChamber X (GenomicTree, Inc., Korea). After hybridization, microarray slides were imaged using Axon 4000B scanner (Axon Instruments Inc., USA). The signal and background fluorescence intensities were calculated for each probe spot by averaging the intensities of every pixel inside the target region using GenePix Pro 4.0 software (Axon Instruments Inc., Foster, Calif., USA). Spots were excluded from analysis due to obvious abnormalities. All data normalization, statistical analysis and cluster analysis were performed using GeneSpring 7.2 (Agilent, USA).

To determine genes showing significant difference in expression level between two groups (group A includes normal and LSIL; and group B includes HSIL, CIS and cancer), statistical analysis (ANOVA test, p<0.05) was performed. 697 genes were identified that were down regulated in group B cells compared with group A cells. The regulated genes in group B were further filtered.

### Example 7

### Identification of Methylation Controlled Gene Expression in Higher Grade Cervical Lesions

To determine whether down regulated genes in cervical lesions are reactivated by demethylation, four cervical cancer cell lines C33A, HeLa, Caski, and SiHa were treated with demethylating agent 5 aza 2′-deoxycytidine (DAC, Sigma)) for 4-5 days at a concentration of 1.0 uM. Cells were harvested and total RNA was isolated from treated and untreated cell lines using Tri reagent. To determine gene expression changes by DAC treatment, transcript level between untreated and treated cell lines was directly compared. 1857 reactivated genes were obtained. 53 common genes between the 697 high grade lesion repressed genes and the 1857 reactivated genes were identified.

### Example 8

### Confirmation of Methylation of Identified Genes

### Example 8.1

### In Silico Analysis of CpG Island in Promoter Region

The promoter regions of the 53 genes were scanned for the presence of CpG islands using MethPrimer (http://itsa.ucsf.edu/˜urolab/methprimer/index1.html). 14 genes did not contain the CpG island and were dropped from the common gene list.

### Example 8.2

### Biochemical Assay for Methylation

To biochemically determine the methylation status of the remaining 39 genes, methylation status of each promoter was detected using the characteristics of restriction endonucleases, HpaII (methylation-sensitive) and MspI (methylation-insensitive) followed by PCR as discussed above in Example 3.2. From this, it was discovered that 11 genes were not methylated, leaving 28 confirmed candidate genes that fit the criteria of being down regulated in high grade lesion, up regulated under demethylation conditions, contains a CpG island in its promoter and is actually methylated in the cancer cell lines.

### Example 8.3

### Bisulfite Sequencing of Methylated Promoter

To further confirm the methylation marker status of the 28 identified genes, the inventors performed bisulfite sequencing of the individual promoters as discussed supra in Example 3.3. The results confirmed that the genes were all methylated.

### Example 9

### Gene expression Profile of the Identified Marker Genes for Higher Grade Lesions

FIG. 10 shows the gene expression profiles of the 28 genes that were identified. As shown in FIG. 10, gene expression was repressed in group B cells (HSIL, CIS and cancer) compared with the group A cells (normal and LSIL).

### Example 10

### Promoter Methylation Assay on Clinical Samples

To determine the clinical applicability of the methylated promoters of the 28 selected genes of the present invention, methylation assay was performed with cervical scrape clinical samples. Methylation assay was performed as described supra using the enzyme digestion/PCR method.

FIG. 11 shows the results of the methylation assay on cervical scrapes as diagnosed with the Bethesda system of cytological indicators. As shown in FIG. 11, most of the genes are not methylated in the normal and LSIL clinical samples but are methylated in HSIL or more severe state cells.

### Example 11

### Promoter Methylation Assay on Cervical Scrapes

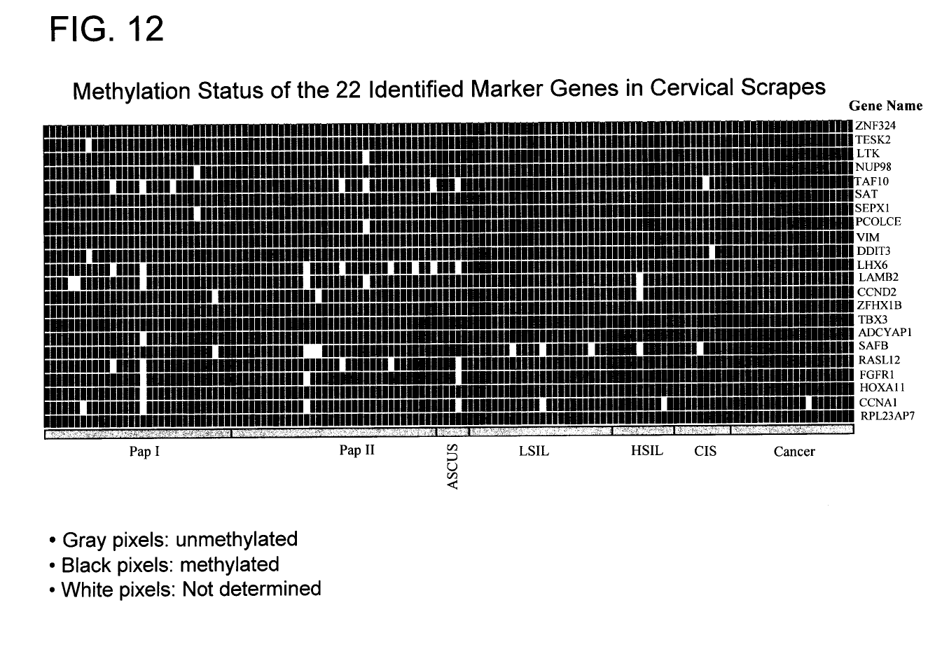

Initially, as seen in FIGS. 6 and 11, a total of 48 markers were identified for diagnosis of cervical cancer. Discrimination of low severity lesions and high severity lesions occurred by two approaches. Methylation assay was performed using cervical scrape samples with the 48 markers (FIG. 6 and 11). Marker genes significantly methylated in cancer or high grade cervical lesions as determined by statistical analysis (ANOVA test, p<0.05) were selected, which narrowed the number of markers to 26 candidate marker genes—16 markers from the 20 marker results (FIG. 6) and 10 markers from the 28 marker results (FIG. 11). The selected 26 marker genes were validated with cervical scrape samples. During initial clinical validation, 4 of these marker genes, GL12, DKFZO047, FGFR1 and ALDH3B1, were determined to be non-specifically methylated irrespective of grade of cervical cytology. Therefore, these 4 markers were excluded in further methylation assays. Methylation assay was performed using 22 markers with 143 clinical cervical scrapes. Except for LAMB2, the remaining 21 biomarkers showed hypermethylation in accordance with the severity of cervical lesions. The 21 biomarkers are listed as follows and are also shown in FIG. 12: ZNF324, TESK2, LTK, NUP98, TAF10, SAT, SEPX1, PCOLCE, VIM, DDIT3, LHX6, CCND2, ZFHX1B, TBX3, ADCYAP1, SAFB, RASL12, FGFR1, HOXA11, CCNA1, RPL23AP7. The descriptions of these genes are set forth previously above.

Based on these results, the invention may be utilized for detecting not only cervical cancer but also the cytological state of these cells. Hence, even low severe (LSIL) cytology can be accurately diagnosed. The accuracy of this method is much greater than using conventional pap smear diagnosis, as false negative results run in the range of about 30-50% of the time. In contrast, using the 21 marker gene profile as shown herein, the diagnostic accuracy can be boosted to a level well over conventional methods.

All of the references cited herein are incorporated by reference in their entirety.

Those skilled in the art will recognize, or be able to ascertain using no more than routine experimentation, many equivalents to the specific embodiments of the invention specifically described herein. Such equivalents are intended to be encompassed in the scope of the claims.

What is claimed is:

1. A method for discovering a methylation marker gene for the conversion of a cell comprising:
(i) comparing converted and unconverted cell gene expression content to identify a gene that is present in greater abundance in the unconverted cell;
(ii) treating a converted cell with a demethylating agent and comparing its gene expression content with gene expression content of an untreated converted cell to identify a gene that is present in greater abundance in the cell treated with the demethylating agent; and
(iii) identifying a gene that is common to the identified genes in steps (i) and (ii), wherein the common identified gene is the methylation marker gene.

2. The method according to claim 1, wherein the comparing is carried out by direct comparison.

3. The method according to claim 1, wherein the comparing is carried out by indirect comparison.

4. The method according to claim 1, wherein the converted cell is cancer cell.

5. The method according to claim 4, wherein the cancer is melanoma, carcinoma, or sarcoma.

6. The method according to claim 5, wherein the cancer is cervical cancer.

7. The method according to claim 1, wherein the converted cell represents cervical dysplasia.

8. The method according to claim 7, wherein the dysplasia is squamous intraepithelial lesion (SIL), low squamous intraepithelial lesion (LSIL), high squamous intraepithelial lesion (HSIL), carcinoma in situ (CIS) or cancer.

9. The method according to claim 1, comprising confirming the methylation marker gene, which comprises assaying for methylation of the common identified gene in the converted cell, wherein the presence of methylation in the promoter region of the common identified gene confirms that the identified gene is the marker gene.

10. The method according to claim 9, wherein the assay for methylation of the identified gene is carried out by
(iv) identifying primers that span a methylation site within the nucleic acid region to be amplified,
(v) treating the genome of the converted cell with a methylation specific restriction endonuclease,
(vi) amplifying the nucleic acid by contacting the genomic nucleic acid with the primers, wherein successful amplification indicates that the identified gene is methylated, and unsuccessful amplification indicates that the identified gene is not methylated.

11. The method according to claim 10, wherein the converted cell genome is treated with an isoschizomer of the methylation sensitive restriction endonuclease that cleaves both methylated and unmethylated CpG-sites as a control.

12. A method of identifying a converted cell comprising assaying for the methylation of the marker gene identified in claim 1.

13. A method of diagnosing cancer or a stage in the progression of the cancer in a subject comprising assaying for the methylation of the marker gene identified using the method in claim 1.

14. The method according to claim 13, wherein the cancer is cervical cancer.

15. The method according to claim 14, wherein the marker gene is Nucleoporin 98 kDa, Selenoprotein X, 1, DKFZP4340047 protein, Zinc finger protein 324, Testis-specific kinase 2, Corin, serine protease, GLI-Kruppel family member GLI2, Spermidine/spermine N1-acetyltransferase, Scaffold attachement factor B, Leucine-rich repeats and calponin homology (CH) domain containing 4, Laminin, beta 2 (Laminin S), ATPase Na+/K+ transporting, beta 2 polypeptide, Tubulin, beta polypeptide, Aldehyde dehydrogenase 3 family, member B1, Leukocyte tyrosine kinase, Procollagen C endopeptiase enhancer, Protein tyrosine phosphatase, receptor type, U, TAF10 RNA polymerase II, TATA box binding protein (TBP)-associated factor 30 kDa, Fibroblast growth factor receptor 1 (fms-related tyrosine kinase 2, Pfeiffer syndrome), DNA-damage-inducible transcript 3, ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin A1; CCND2 (NT_{—}009759): Cyclin D2; EPHA5 (NT_{—}022778): EphA5; HOXA11 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT_{—}086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin, and combinations thereof.

16. A method of diagnosing high grade lesion of cervical dysplasia comprising assaying for methylation of a marker gene as follows: ADCYAP1 (NT_{—}010859): Adenylate cyclase activating polypeptide 1 (pituitary); C10orf116 (NT_{—}030059): Chromosome 10 open reading frame 116; CCNA1 (NT_{—}024524): Cyclin A1; CCND2 (NT_{—}009759): Cyclin D2; EPHA5 (NT_{—}022778): EphA5; HOXA11 (NT_{—}007819): Homeo box A11; IGFBP4 (NT_{—}010755): Insulin-like growth factor binding protein 4; KIAA1467 (NT_{—}009714); LHX6 (NT_{—}008470): LIM homeobox 6; MAL (NT_{—}026970): Mal, T-cell differentiation protein; MRC2 (NT_{—}010783): Mannose receptor, C type 2; RASL12 (NT_{—}010194): RAS-like, family 12; RPL23AP7 (MGC70863, NT_{—}011526): SIMILAR TO RIBOSOMAL PROTEIN L23A; SLC30A3 (NT_{—}022184): Solute carrier family 30 (zinc transporter), member 3; TBX3 (NT_{—}009775): T-box 3 (ulnar mammary syndrome); VIM (NT_{—}077569): Vimentin; ZFHX1B (NT_{—}005058); ZNF486 (NT_{—}011295); CD34 (NT_{—}021877): CD34 antigen; CDC34 (NT_{—}011255): Cell division cycle 34; CTF1 (NT_{—}086679): Cardiotrophin 1; CX3CR1 (NT_{—}022517): Chemokine (C-X3-C motif) receptor 1; FDPS (NT_{—}004487): FARNESYL PYROPHOSPHATE SYNTHETASE (FPPS); GSTM4 (NT_{—}019273): Glutathione S-transferase M4; MYH7B (NT_{—}028392): MYOSIN, HEAVY POLYPEPTIDE 7B, CARDIAC MU; SEC61A2 (NT_{—}077569): Sec61 alpha 2 subunit (S. cerevisiae); STOML1 (NT_{—}010194): Stomatin (EPB72)-like 1; and THBD (NT_{—}011387): Thrombomodulin or a combination thereof

17. The method according to claim 16, wherein high grade lesion is intraepithelial lesion (HSIL), carcinoma in situ (CIS) or cancer.

18. The method according to claim 16, wherein the dysplasia is observed in sample taken from scrape, biopsy, blood or urine.

19. A method of diagnosing high grade lesion of cervical dysplasia comprising assaying for methylation of a marker gene as follows: ZNF324, TESK2, LTK, NUP98, TAF10, SAT, SEPX1, PCOLCE, VIM, DDIT3, LHX6, CCND2, ZFHX1B, TBX3, ADCYAP1, SAFB, RASL12, FGFR1, HOXA11, CCNA1, RPL23AP7.
